(19) United States
(12) Patent Application Publication
(10) Pub. No.: US 2005/0055010 A1
(43) Pub. Date: Mar. 10, 2005

(54) Method for altering insulin pharmacokinetics

(76) Inventors: Ronald J. Pettis, Durham, NC (US); Noel Harvey, Efland, NC (US); Barry Ginsberg, Wyckoff, NJ (US)

Correspondence Address: JONES DAY, 222 EAST 41ST ST, NEW YORK, NY, 10017, US

(21) Appl. No.: 10/841,992

(22) Filed: May 6, 2004

Related U.S. Application Data
(60) Provisional application No. 60/523,831, filed on Nov. 19, 2003.
(60) Provisional application No. 60/500,956, filed on Sep. 5, 2003.

(51) Int. Cl. A61K 38/28; A61M 31/00

(52) U.S. Cl. 604/500; 514/3

(57) ABSTRACT

The present invention relates to methods for administration of insulin into the intradermal compartment of subject's skin, preferably to the dermal vasculature of the intradermal compartment. The methods of the present invention enhance the pharmacokinetic and pharmacodynamic parameters of insulin delivery and effectively result in a superior clinical efficacy in the treatment and/or prevention of diabetes mellitus. The methods of the instant invention provide an improved glycemic control of both non-fasting (i.e., post-prandial) and fasting blood glucose levels and thus have an enhanced therapeutic efficacy in treatment, prevention and/or management of diabetes relative to traditional methods of insulin delivery, including subcutaneous insulin delivery.

This application claims priority to U.S. application Ser. No. 10/429,973, filed on May 6, 2003, which claims priority to U.S. Provisional applications Nos. 60/377,649 and 60/389,888 filed May 6, 2002 and Jun. 20, 2002, respectively all of which are incorporated herein by reference in their entireties. This application additionally claims priority to U.S. Provisional Application Nos. 60/523,831 and 60/500,956 filed on Nov. 19, 2003 and Sep. 5, 2003, respectively, all of which are incorporated herein by reference in their entireties.

The following description includes information that may be useful in understanding the present invention. It is not an admission that any of the information provided herein is prior art to the presently claimed inventions, or relevant, nor that any of the publications specifically or implicitly referenced are prior art.

### 1. FIELD OF THE INVENTION

The present invention relates to methods for administration of insulin into the intradermal compartment of subject's skin, preferably to the dermal vasculature of the intradermal compartment. The methods of the present invention enhance the pharmacokinetic and pharmacodynamic parameters of insulin delivery and effectively result in a superior clinical efficacy in the treatment and/or prevention of diabetes mellitus. The methods of the instant invention provide an improved glycemic control of both non-fasting (i.e., post-prandial) and fasting blood glucose levels and thus have an enhanced therapeutic efficacy in treatment, prevention and/or management of diabetes relative to traditional methods of insulin delivery, including subcutaneous insulin delivery.

### 2. BACKGROUND OF THE INVENTION

2.1. Drug Delivery

The importance of efficiently and safely administering pharmaceutical substances such as diagnostic agents and drugs has long been recognized. Although an important consideration for all pharmaceutical substances, obtaining adequate bioavailability of large molecules such as proteins that have arisen out of the biotechnology industry has recently highlighted this need to obtain efficient and reproducible absorption (Cleland et al., 2001 Curr. Opin. Biotechnol. 12: 212-219). The use of conventional needles has long provided one approach for delivering pharmaceutical substances to humans and animals by administration through the skin. Considerable effort has been made to achieve reproducible and efficacious delivery through the skin while improving the ease of injection and reducing patient apprehension and/or pain associated with conventional needles. Furthermore, certain delivery systems eliminate needles entirely, and rely upon chemical mediators or external driving forces such as iontophoretic currents or electroporation or thermal poration or sonophoresis to breach the stratum corneum, the outermost layer of the skin, and deliver substances through the surface of the skin. However, such delivery systems do not reproducibly breach the skin barriers or deliver the pharmaceutical substance to a given depth below the surface of the skin and consequently, clinical results can be variable. Thus, mechanical breach of the stratum corneum such as with needles, is believed to provide the most reproducible method of administration of substances through the surface of the skin, and to provide control and reliability in placement of administered substances.

Approaches for delivering substances beneath the surface of the skin have almost exclusively involved transdermal administration, i.e., delivery of substances through the skin to a site beneath the skin. Transdermal delivery includes subcutaneous, intramuscular or intravenous routes of administration of which, intramuscular (IM) and subcutaneous (SC) injections have been the most commonly used.

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University Press, New York, 1991). The epidermis is subdivided into five layers or strata of a total thickness of between 75 and 150 μm. Beneath the epidermis lies the dermis, which contains two layers, an outermost portion referred to as the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue.

As noted above, both the subcutaneous tissue and muscle tissue have been commonly used as sites for administration of pharmaceutical substances. The dermis, however, has rarely been targeted as a site for administration of substances, and this may be due, at least in part, to the difficulty of precise needle placement into the intradermal space. Furthermore, even though the dermis, in particular, the papillary dermis has been known to have a high degree of vascularity, prior to the instant invention it was not appreciated that one could take advantage of this high degree of vascularity to obtain an improved absorption profile for administered substances compared to subcutaneous administration.

Small drug molecules have been traditionally administered subcutaneously because they are rapidly absorbed after administration into the subcutaneous tissue and subcutaneous administration provides an easy and predictable route of delivery. However, the need for improving the pharmacokinetics of administration of small molecules has not been appreciated. Large molecules such as proteins are typically not well absorbed through the capillary epithelium regardless of the degree of vascularity of the targeted tissue. Effective subcutaneous administration for these substances has thus been limited.

One approach to administration beneath the surface to the skin and into the region of the intradermal space has been routinely used in the Mantoux tuberculin test. In this procedure, a purified protein derivative is injected at a shallow angle to the skin surface using a 27 or 30 gauge needle (Flynn et al., 1994 Chest 106:1463-5). A degree of uncertainty in placement of the injection can, however, result in some false negative test results. Moreover, the test has involved a localized injection to elicit a response at the site of injection and the Mantoux approach has not led to the use of intradermal injection for systemic administration of substances.

Some groups have reported on systemic administration by what has been characterized as “intradermal” injection. In one such report, a comparative study of subcutaneous and what was described as “intradermal” injection was performed (Autret et al., 1991 Therapie 46:5-8). The pharmaceutical substance tested was calcitonin, a protein of a molecular weight of about 3600. Although it was stated that the drug was injected intradermally, the injections used a 4 mm needle pushed up to the base at an angle of 60. This would have resulted in placement of the injectate at a depth of about 3.5 mm and into the lower portion of the reticular dermis or into the subcutaneous tissue rather than into the vascularized papillary dermis. If, in fact, this group injected into the lower portion of the reticular dermis rather than into the subcutaneous tissue, it would be expected that the substance would either be slowly absorbed in the relatively less vascular reticular dermis or diffuse into the subcutaneous region to result in what would be functionally the same as subcutaneous administration and absorption. Such actual or functional subcutaneous administration would explain the reported lack of difference between subcutaneous and what was characterized as intradermal administration, in the times at which maximum plasma concentration was reached, the concentrations at each assay time and the areas under the curves.

Similarly, Bressolle et al. administered sodium ceftazidime in what was characterized as “intradermal” injection using a 4 mm needle (Bressolle et al., 1993 J. Pharm. Sci. 82:1175-1178). This would have resulted in injection to a depth of 4 mm below the skin surface to produce actual or functional subcutaneous injection, although good subcutaneous absorption would have been anticipated in this instance because sodium ceftazidime is hydrophilic and of relatively low molecular weight.

Another group reported on what was described as an intradermal drug delivery device (U.S. Pat. No. 5,007,501). Injection was indicated to be at a slow rate and the injection site was intended to be in some region below the epidermis, i.e., the interface between the epidermis and the dermis or the interior of the dermis or subcutaneous tissue. This reference, however, provided no teachings that would suggest a selective administration into the dermis nor did the reference suggest any possible pharmacokinetic advantage that might result from such selective administration.

Thus, there remains a continuing need for efficient and safe methods and devices for administration of pharmaceutical substances.

2.2. Diabetes Mellitus

Diabetes mellitus is characterized by a broad array of physiologic and anatomic abnormalities, for example, abnormal insulin secretion, altered glucose disposition, altered metabolism of lipid, carbohydrates, and proteins, hypertension, neuropathy, retinopathy, abnormal platelet activity, and an increased risk of complications from vascular disease. Diabetics are generally divided into two categories. Patients who depend on insulin for the prevention of ketoacidosis have insulin-dependent diabetes mellitus (IDDM) or type 1 diabetes. Diabetics who do not depend on insulin to avoid ketoacidosis have non-insulin-dependent diabetes mellitus (NIDDM) or type 2 diabetes.

Diabetes is typically classified further into two categories: primary and secondary. Primary diabetes includes Insulin-dependent diabetes mellitus (IDDM Type 1), Non-insulin-dependent diabetes mellitus (NIDDM Type 2) which further includes Nonobese NIDDM, Obese NIDDM and Maturity-onset diabetes of the young. Primary diabetes implies that no associated disease is present, while in secondary diabetes some other identifiable condition causes or allows a diabetic syndrome to develop. Examples of diabetic syndromes that may contribute to the development of secondary diabetes include pancreatic disease, hormonal abnormalities, drug or chemical induced conditions, and genetic syndromes.

Insulin dependence in this classification is not equivalent to insulin therapy, but means that the patient is at risk for ketoacidosis in the absence of insulin. It has been suggested that the terms insulin-dependent and non-insulin-dependent describe physiologic states (ketoacidosis-prone and ketoacidosis-resistant, respectively), while the terms Type 1 and Type 2 refer to pathogenetic mechanisms (immune-mediated and non-immune-mediated, respectively). Using this classification, three major forms of primary diabetes are recognized: (1) type 1 insulin-dependent diabetes [IDDM], (2) type 2 non-insulin-dependent diabetes [NIDDM], and (3) gestational diabetes. Secondary forms of diabetes encompass a host of conditions such as pancreatic disease, hormonal abnormalities, genetic syndromes, and others.

Insulin-dependent diabetes mellitus often develops in childhood or adolescence while the onset of NIDDM generally occurs in middle or late life. Patients with NIDDM are usually overweight and constitute 90 to 95 percent of all diabetics. IDDM results from the destruction of beta cells by an autoimmune process that may be precipitated by a viral infection. NIDDM is characterized by a gradual decline in beta cell function and varying degrees of peripheral resistance to insulin. The annual incidence of IDDM ranges from 10 cases per 100,000 persons for nonwhite males to 16 cases per 100,000 persons for white males (LaPorte et al., 1981, Diabetes 30: 279). The prevalence of NIDDM increases with age, especially after age 45 and is higher among blacks than whites and certain populations such as Asian Indians living in South Africa and England (Malter et al., 1985, Br. Med. J. 291: 1081). Gestational diabetes occurs in 2.4 percent of all pregnancies in the United States annually (Freinkel et al., 1985, N. Engl. J. Med. 313: 96). Pregnancy is also a state of insulin resistance. This insulin resistance is exacerbated in gestational diabetes which may predispose patients to the various hypertensive syndromes of pregnancy associated with NIDDM (Bardicef et al., 1995, Am. J. Gynecol. 172:1009-1013).

Current therapies for IDDM include insulin therapy, and for NIDDM will include dietary modification in a patient who is overweight and hypoglycemic agents, e.g., glipizide, glyburide and gliperimide, all of which act by stimulating the release of insulin from the beta cells and metformin, and thiazolidinediones which reduce insulin resistance. However, there is still an unmet need for effective insulin therapy with optimal pharmacokinetic parameters.

### 3. SUMMARY OF THE INVENTION

The present invention relates to an improved parenteral administration method for delivering insulin to a subject, preferably humans, by directly targeting the dermal space whereby such method dramatically alters the pharmacokinetics (PK) and pharmacodynamics (PD) parameters of the administered insulin. The altered PK and PD parameters enhance the therapeutic efficacy of the administered insulin. Thus, the methods of the invention are particularly useful for the treatment, prevention and/or management of diabetes mellitus such as insulin-dependent diabetes mellitus and/or non-insulin dependent diabetes mellitus. The methods of the invention ameliorate one or more symptoms associated with diabetes mellitus.

Intradermal delivery of insulin in accordance with the methods of the invention provides an improved glycemic control and thus has an enhanced therapeutic efficacy in treatment, prevention and/or management of diabetes relative to traditional methods of insulin delivery, including subcutaneous insulin delivery. Preferably, the methods of the invention provide an improved glycemic control without an increase in hypoglycemic events. Although not intending to be bound by a particular mechanism of action, the improved glycemic control achieved using the intradermal delivery methods of the invention is due, in part, to the control of both non-fasting (i.e., post prandial) and fasting glucose levels. The intradermal delivery methods of the invention lower fasting and/or post-prandial hyperglycemia more effectively than traditional methods of insulin delivery.

Intradermal delivery of insulin in accordance with the methods of the invention is particularly useful in controlling post-prandial hyperglycemia. As used herein, “post-prandial” carries its ordinary meaning in the art and refers to plasma glucose concentrations after eating a meal (e.g., a non-fasted state), and is often measured 2 hours after the meal (i.e., 2 hour post-prandial glucose). The intradermal delivery methods of the invention effectively control post-prandial glucose levels within the first two hours, preferably within the first hour after insulin delivery. Although not intending to be bound by a particular mechanism of action, intradermal insulin delivery in accordance with the methods of the invention results in effective systemic absorption of insulin within the first hour which results in reduction of post-prandial glucose (PPG) levels. Preferably, insulin delivery results in reduction of PPG levels by at least 20 mg/dL, at least 30 mg/dL, at least 40 mg/dL or at least 50 mg/dL. In a preferred embodiment, intradermal insulin delivery in accordance with the methods of the invention results in a reduction of PPG levels by 45 mg/dL.

Insulin delivered in accordance with the methods of the invention results in a higher biopotency relative to traditional methods of insulin delivery, including subcutaneous insulin delivery. Biopotency in general refers to the strength of a chemical substance on the body, and how well or how far it can act on a biological system. Biopotency as used herein refers to how well or how far insulin can act on a biological system and includes its ability to affect glycemic control, including fasting blood glucose levels and post-prandial glucose levels. Although not intending to be bound by a particular mechanism of action, the increased biopotency of insulin delivered in accordance with the methods of the invention is due, in part, to being systemically absorbed rapidly within the first hour of delivery.

The invention encompasses methods of administering solution forms of insulin (e.g., Humalog®), particulate forms of insulin, and mixtures thereof (e.g., Humalog® Mix 50/50™). The insulin formulations may be in different physical association states, including but not limited to monomeric, dimeric and hexameric states. The chemical state of insulin may be modified by standard recombinant DNA technology to produce insulin of different chemical formulas in different association states. Alternatively, solution parameters, such as pH and Zn content, may be altered to result in formulations of insulin in different association states. Other chemical modifications of insulin or addition of additives or excipients to alter absorption of insulin are also encompassed by the instant invention.

As used herein, intradermal administration is intended to encompass administration of insulin into the dermis in such a manner that the substance readily reaches the dermal vasculature, including both the circulatory and lymphatic vasculature, and is rapidly absorbed into the blood capillaries and/or lymphatic vessels to become systemically bioavailable. It is believed that deposition of a substance predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption of insulin. Preferably, insulin is delivered in accordance with the present invention at a depth of 1.75 mm, 1.5 mm or 1.25 mm.

Directly targeting the dermal space, preferably the dermal vasculature, as taught by the invention provides more rapid onset of effects of insulin. The inventors have found that insulin can be rapidly absorbed and systemically distributed via controlled ID administration that selectively accesses the circulatory and lymphatic microcapillaries, thus insulin may exert their beneficial effects more rapidly than SC administration. The methods of the invention better facilitate some current therapies such as blood glucose control via insulin delivery.

Delivering insulin to the intradermal compartment, preferably the dermal vasculature, results in improved pharmacokinetics relative to conventional methods of insulin delivery. According to the present invention, improved pharmacokinetics means increased bioavailability, decreased lag time (T_{lag}), decreased Tₘₐₓ, more rapid absorption rates, more rapid onset and/or increased Cₘₐₓ for a given amount of compound administered, compared to conventional insulin delivery. By bioavailability is meant the total amount of a given dosage of the delivered substance that reaches the blood compartment. This is generally measured as the area under the curve in a plot of concentration vs. time. By “lag time” is meant the delay between the administration of the delivered substance and time to measurable or detectable blood or plasma levels. Tₘₐₓ is a value representing the time to achieve maximal blood concentration of the compound, and Cₘₐₓ is the maximum blood concentration reached with a given dose and administration method. The time for onset is a function of T_{lag}, Tₘₐₓ and Cₘₐₓ, as all of these parameters influence the time necessary to achieve a blood (or target tissue) concentration necessary to realize a biological effect. Tₘₐₓ and Cₘₐₓ can be determined by visual inspection of graphical results and can often provide sufficient information to compare two methods of administration of a compound. However, numerical values can be determined more precisely by kinetic analysis using mathematical models and/or other means known to those of skill in the art.

In some embodiments, delivery of insulin is done in a controlled manner, e.g., by controlling the volume of delivery to achieve a monophasic pharmacokinetic profile, e.g., a kinetic profile wherein the drug concentration vs. time profile can be mathematically fit using only one mode or route of absorption and distribution, preferably intradermal.

Furthermore, it was unexpectedly discovered that, when mixtures of particulate and solution forms of insulin are administered according to the methods of the invention, it is possible to achieve a prolonged circulation of insulin, while retaining the rapid onset of systemic availability of insulin. Therefore, a particular advantage of the methods of the invention is an improved pharmacokinetic profile of insulin, wherein the pharmacokinetic profile resembles that of a biphasic (or multiphasic) mode of delivery, (i.e., the PK profile can be mathematically fit using two or more modes or routes of absorption and distribution), and will exhibit both an initial or early phase characterized by rapid and high peak onset of insulin levels, followed by a later phase characterized by lower prolonged circulating levels of insulin over a more extended duration.

In accordance with the invention direct intradermal (ID) administration can be achieved using, for example, microneedle-based injection and infusion systems or any other means known to one skilled in the art to accurately target the intradermal space. Particular devices include those disclosed in WO 01/02178, published Jan. 10, 2002; and WO 02/02179, published Jan. 10, 2002, U.S. Pat. No. 6,494,865, issued Dec. 17, 2002 and U.S. Pat. No. 6,569,143 issued May 27, 2003 all of which are incorporated herein by reference in their entirety, as well as those exemplified in FIGS. 8-10. Using the methods of the invention, the pharmacokinetics of insulin, can be altered when compared to traditional methods of insulin delivery. Improved pharmacokinetic parameters using methods of the invention can be achieved using not only microdevice-based injection systems, but other delivery systems such as needle-less or needle-free ballistic injection of fluids or powders into the ID space, Mantoux-type ID injection, enhanced ionotophoresis through microdevices, and direct deposition of fluid, solids, or other dosing forms into the skin.

Another benefit of the invention is to achieve more rapid systemic distribution and offset of insulin. The methods of the invention also help achieve higher bioavailabilities of insulin. The direct benefit is that ID administration with enhanced bioavailability allows equivalent biological effects while using less active agent. This results in direct economic benefit to the drug manufacturer and perhaps consumer. Likewise, higher bioavailability may allow reduced overall dosing and decrease the patient's side effects associated with higher dosing. The more rapid offset of insulin may produce a decreased rate of hypoglycemia.

Yet another benefit of the invention is the attainment of higher maximum concentrations of insulin in the plasma. The inventors have found that insulin administered in accordance with the methods of the invention is absorbed more rapidly, resulting in higher initial concentrations in the plasma. The more rapid onset allows higher C_{Max} values to be reached with lesser amounts of insulin.

Another benefit of the invention is removal of the physical or kinetic barriers invoked when insulin passes through and becomes trapped in cutaneous tissue compartments prior to systemic absorption. Direct ID administration by mechanical means in contrast to transdermal delivery methods overcomes the kinetic barrier properties of skin, and is not limited by the pharmaceutical or physicochemical properties of insulin or its formulation excipients.

These and other benefits of the invention are achieved by directly targeting the dermal vasculature and by controlled delivery of insulin to the dermal space of skin. The inventors have found that by specifically targeting the intradermal space and controlling the rate and pattern of delivery, the pharmacokinetics exhibited by insulin can be unexpectedly improved, and can in many situations be varied with resulting clinical advantage. Such pharmacokinetic control cannot be as readily obtained or controlled by other parenteral administration routes, except by IV access.

Using the methods of the present invention, insulin may be administered as a bolus, or by infusion. As used herein, the term “bolus” is intended to mean an amount that is delivered within a time period of less than ten (10) minutes. “Infusion” is intended to mean the delivery of a substance over a time period greater than ten (10) minutes. It is understood that bolus administration or delivery can be carried out with rate controlling means, for example a pump, or have no specific rate controlling means, for example user self-injection.

The insulin formulations of the invention may be in any form suitable for intradermal delivery. In one embodiment, the intradermal insulin formulation of the invention is in the form of a flowable, injectible medium, i.e., a low viscosity formulation that may be injected in a syringe. The flowable injectible medium may be a liquid. Alternatively, the flowable injectible medium is a liquid in which particulate material is suspended, such that the medium retains its fluidity to be injectible and syringable, e.g., can be administered in a syringe. The invention encompasses formulations in which insulin is in a particulate form, i.e., is not fully dissolved in solution. In some embodiments, at least 30%, at least 50%, at least 75% of the insulin is in particulate form. Although not intending to be bound by a particular mode of action, formulations of the invention in which insulin is in particulate form have at least one agent which facilitates the precipitation of insulin. Precipitating agents that may be employed in the formulations of the invention may be proteinacious, e.g., protamine, a cationic polymer, or non-proteinacious, e.g., zinc or other metals or polymers.

In a specific embodiment, the insulin formulation administered in accordance with the methods of the invention is Insulin Lispro (Eli Lilly & Company) at 100 U/mL. Preferably 1 to 50 U, most preferably 10 U, of Insulin Lispro are used in the methods of the invention. In another specific embodiment, the insulin formulation administered in accordance with the methods of the invention is 20 U 50% pre-mixed insulin Lispro (Humalog Mix 50/50™, containing 50% insulin Lispro and 50% insulin Lispro protamine suspension).

Insulin can be formulated at any solution concentration ranging from 10 International Units/mL, up to, and including, 500 International Units/mL. The invention preferably encompasses administering 1 to 50U of insulin formulations as disclosed herein. Using the methods of the invention lower doses of insulin are required to achieve a similar therapeutic efficacy as conventional methods of insulin therapy. The insulin formulations delivered in accordance with the methods of the invention are particularly effective in decreasing serum glucose levels and have improved therapeutic efficacy compared to the conventional methods for treating and/or preventing diabetes mellitus.

The intradermal insulin formulations of the present invention can be prepared as unit dosage forms. A unit dosage per vial may contain 0.1 to 0.5 mL of the formulation. In some embodiments, a unit dosage form of the intradermal formulations of the invention may contain 50 μL to 100 μL, 50 μL to 200 μL, or 50 μL to 500 μL of the formulation. If necessary, these preparations can be adjusted to a desired concentration by adding a sterile diluent to each vial.

The present invention improves the clinical utility of ID delivery of insulin to humans or animals. The clinical utility of ID delivery is improved by delivering to the intradermal compartment, preferably the dermal vasculature. Disclosed is a method to increase the rate of uptake for insulin without necessitating SC access. This effect provides a shorter Tₘₐₓ. Potential corollary benefits include higher maximum concentrations for a given unit dose (Cₘₐₓ), higher bioavailability, more rapid onset of pharmacodynamics or biological effects, and reduced depot effects.

### 4. DESCRIPTION OF THE DRAWINGS

FIG. 1 PHARMACOKINETIC PROFILE OF INSULIN LISPRO DELIVERED ID VS. SC. Insulin Lispro levels over time after delivery of insulin into skin at three different ID depths are shown and compared to the profile obtained with SC delivery. For SC injection, a 30 Ga, 8 mm standard insulin syringe and needle were used with a pinch up technique.

FIG. 2 BIOAVAILABILITY OF INSULIN LISPRO. This bar graph shows bioavailability upon ID administration of insulin to either 1.25 mm, 1.5 mm (result in duplicate), 1.75 mm depth, or SC administration of insulin. The absolute AUC is shown in light grey; and the % AUC is shown in dark grey.

FIGS. 3A and B PHARMACODYNAMIC PROFILE OF HUMALOG. The glucose infusion rate needed in a euglycemic glucose clamp in the average of 10 subjects is shown. Panel A is the raw data and Panel B the filled curve. Done

FIG. 4 PROFILES OF INSULIN HUMALOG® 50/50 MIX. Plasma insulin levels of Humalog Mix 50/50™ containing 50% insulin Lispro and 50% insulin Lispro protamine suspension delivered ID at a depth of 1.5 mm were compared to insulin delivered SC.

FIG. 5 PHARMACODYNAMIC PROFILE OF INTRADEMAL HUMALOG® 50/50 MIX. Blood glucose needed in a glucose clamp in response to levels of Humalog® Mix 50/50™ containing 50% insulin Lispro and 50% insulin Lispro protamine suspension delivered ID at a depth of 1.5 mm were compared to insulin delivered SC.

FIG. 6 EFFECT OF ID DELIVERY OF INSULIN ON POST-PRANDIAL BLOOD GLUCOSE. Post-prandial glucose levels were calculated based upon data from the pharmacokinetics and pharmacodynamics after intradermal delivery of insulin Lispro with a 1.5 mm needle.

FIG. 7 ANALYSIS OF THE INCREASE IN EARLY INSULIN LEVELS: COMPARISON OF ID AND SC DELIVERY. Insulin Lispro levels over time were calculated for ID and SC delivery. Data from insulin Lispro that was delivered into skin at an ID depth of 1.5 mm are presented. For SC injection, a 30 G, 8 mm standard insulin syringe and needle were used with a pinch up technique.

FIG. 8 NEEDLE DEVICE. An exploded, perspective illustration of a needle assembly designed according to this invention.

FIG. 9 NEEDLE DEVICE. A partial cross-sectional illustration of the embodiment in FIG. 8.

FIG. 10 NEEDLE DEVICE. Embodiment of FIG. 9 attached to a syringe body to form an injection device.

### 5. DETAILED DESCRIPTION OF THE INVENTION

The present invention provides a method for treatment and/or prevention of diabetes mellitus such as insulin-dependent diabetes mellitus and/or non-insulin dependent diabetes mellitus by delivery of insulin to a mammal, preferably a human by directly targeting the intradermal space, where insulin is administered to the intradermal space. In some embodiments, insulin is deposited to the upper region of the dermis (i.e., the dermal vasculature). Once insulin is infused according to the methods of the invention to the dermal vasculature it exhibits pharmacokinetics superior to, and more clinically desirable than that observed for insulin administered by conventional methods of insulin delivery, e.g., SC injection.

While not intending to be bound by any theoretical mechanism of action, it is believed that the rapid absorption observed upon administration into the dermal vasculature is achieved as a result of the rich plexuses of blood and lymphatic vessels therein. One possible explanation for the unexpected enhanced absorption reported herein is that upon injection of insulin so that it readily reaches the dermal vasculature, an increase in blood flow and capillary permeability results. For example, it is known that a pinprick insertion to a depth of 3 mm produces an increase in blood flow and this has been postulated to be independent of pain stimulus and due to tissue release of histamine (Arildsson et al., 2000 Microvascular Res. 59:122-130). This is consistent with the observation that an acute inflammatory response elicited in response to skin injury produces a transient increase in blood flow and capillary permeability (see, Physiology, Biochemistry, and Molecular Biology of the Skin, Second Edition, L. A. Goldsmith, Ed., Oxford Univ. Press, New York, 1991, p. 1060; Wilhem, Rev. Can. Biol. 30:153-172, 1971). At the same time, the injection into the intradermal layer would be expected to increase interstitial pressure. It is known that increasing interstitial pressure from values (beyond the “normal range”) of about −7 to about +2 mm Hg distends lymphatic vessels and increases lymph flow (Skobe et al., 2000 J. Investig. Dermatol. Symp. Proc. 5:14-19). Thus, the increased interstitial pressure elicited by injection into the intradermal layer is believed to elicit increased lymph flow and increased absorption of substances injected into the dermis.

Intradermal delivery of insulin in accordance with the methods of the invention provides an improved glycemic control and thus has an enhanced therapeutic efficacy in treatment, prevention and/or management of diabetes relative to traditional methods of insulin delivery, including subcutaneous insulin delivery. Preferably, the methods of the invention provide an improved glycemic control without an increase in hypoglycemic events. Although not intending to be bound by a particular mechanism of action, the improved glycemic control achieved using the intradermal delivery methods of the invention is due, in part, to control of both non-fasting (i.e., post-prandial) and fasting glucose levels. The intradermal delivery methods of the invention lower fasting and/or post-prandial hyperglycemia more effectively than traditional methods of insulin delivery.

Intradermal delivery of insulin in accordance with the methods of the invention is particularly useful in controlling post-prandial hyperglycemia. As used herein, “post-prandial” carries its ordinary meaning in the art and refers to plasma glucose concentrations after eating a meal (e.g., a non-fasting state). In non-diabetic individuals, fasting plasma glucose concentrations, e.g., following an overnight 8 to 10 hour fast, generally ranges from 70 to 110 mg/dL. Glucose concentrations begin to rise about 10 min after a meal as a result of absorption of dietary carbohydrates. The post-prandial glucose (PPG) profile is thus determined by carbohydrate absorption, insulin and glucagon secretion, and their coordinated effects on glucose metabolism in the liver and peripheral tissues. The magnitude and time of the peak of plasma glucose concentration depends on various factors including, but not limited to, timing, quantity and composition of the meal. In non-diabetic individuals, plasma glucose concentrations peak about 60 min after start of a meal and rarely exceed 140 mg/dL, and return to pre-prandial levels within 2-3 hours. In diabetic individuals, e.g., patients with type 1 diabetes, who have no endogenous insulin secretion, the time and height of peak insulin concentration and resultant glucose levels are dependent on the amount, type, and route of insulin administration In type 2 diabetes peak insulin levels are delayed and are insufficient to control PPG levels. Furthermore, in type 1 and type 2 diabetic patients additional complications such as abnormalities in insulin and glucagon secretion, hepatic glucose uptake, suppression of hepatic glucose production, and peripheral glucose uptake contribute to higher and more prolonged PPG excursions, i.e., change in glucose concentration from before to after a meal, than in non-diabetic individuals. Therefore, elevated PPG concentrations contribute to suboptimal glucose control.

The intradermal delivery methods of the invention effectively control post-prandial glucose levels within the first two hours, preferably within the first hour after insulin delivery. Although not intending to be bound by a particular mechanism of action, intradermal insulin delivery in accordance with the methods of the invention results in effective systemic absorption of insulin within the first hour which results in reduction of PPG levels. Preferably, insulin delivery results in reduction of PPG levels by at least 20 mg/dL, at least 30 mg/dL, at least 40 mg/dL or at least 50 mg/dL. In a preferred embodiment, intradermal insulin delivery in accordance with the methods of the invention results in a reduction of PPG levels by 45 mg/dL.

Insulin delivered in accordance with the methods of the invention results in a higher biopotency relative to traditional methods of insulin delivery, including subcutaneous insulin delivery. Insulin delivery in accordance with the methods of the invention results in at least 30%, at least 40%, at least 50%, at least 60%, at least 70%, or at least 80% higher biopotency relative to traditional methods of insulin delivery. Biopotency as used herein refers to how well or how far insulin can act on a biological system and includes its ability to affect glycemic control, including fasting blood glucose levels, post-prandial glucose levels and the rate of utilization of glucose by the body. Although not intending to be bound by a particular mechanism of action, the increased biopotency of insulin delivered in accordance with the methods of the invention is due in part to being absorbed rapidly within the first hour.

In a preferred embodiment, the methods of the invention control post-prandial glucose level and thus prevent or delay the onset of microvascular or macrovascular complications caused by diabetes, including but not limited to coronary heart disease, myocardial infarcation, stroke, retinopathy, neuropathy and renal failure. Although not intending to be bound by a particular mechanism of action, post prandial hyperglycemia is associated with endothelial dysfunction and one of the first steps in atherogenesis.

Furthermore, it was unexpectedly discovered that, when mixtures of particulate and solution forms of insulin are administered according to the methods of the invention, it is possible to achieve a prolonged circulation of insulin, while retaining the rapid onset of systemic availability of insulin. Without being limited by a particular theory, while the solution form of insulin, when intradermally administered, contributes to the rapid onset of systemic availability of insulin, the particulate form of insulin is not systemically immediately available in a biologically active form. Without being limited by a theory, as the precipitating agent (e.g., protamine), which is present in the particulate formulation of insulin, diffuses away, insulin gradually becomes resolubilized in the solution, systemically circulated over a prolonged period of time. Accordingly, this invention encompasses methods of eliciting a prolonged circulation of insulin, while eliciting a more rapid onset of systemic availability of insulin than subcutaneous delivery, in a human subject, comprising delivering into an intradermal compartment of the human subject's skin an insulin formulation which comprises both particulate and solubilized forms of insulin.

As used herein, and unless otherwise specified, the term “prolonged circulation” means that the circulation half life of insulin, delivered using methods of the invention, is longer than the circulation half life of insulin delivered using other methods of intradermal delivery (e.g., intradermal delivery of solution form of insulin). Moreover, the term also denotes that the circulation half life of insulin, delivered using methods of the invention, is at least comparable to, or longer than, that of insulin delivered into other compartments (e.g., subcutaneous).

In other embodiments, the rate of release of insulin can be controlled by varying the ratio between the particulate and solution forms of insulin contained in the formulation to be administered using methods of the invention. Therefore, this invention also encompasses methods of modulating circulation half life of insulin in a human subject, comprising administering into an intradermal compartment of the human subject's skin a composition comprising both particulate and solution forms of insulin, wherein the ratio between the particulate and solution forms of insulin is varied. Methods of the invention thus provide a controlled means of modulating circulation half life of insulin, while achieving a rapid onset of systemic availability at the same time.

Furthermore, circulating half lives of other therapeutic agents, particularly protein-based therapeutic agents, can be similarly controlled using methods of this invention, while enhancing their systemic availability by enhancing their onset. Methods of the invention are particularly preferred for extended release formulations. Thus, in other embodiments, this invention encompasses methods of modulating circulation half life of a therapeutic agent in a human subject, comprising administering into an intradermal compartment of the human subject's skin a composition comprising both particulate and solution forms of the therapeutic agent, wherein the ratio between the particulate and solution forms of the therapeutic agent is varied. In a particular embodiment, the therapeutic agent is a protein. Methods of the invention are particularly preferred for pain medications, oncological agents such as interferons, growth hormones, protein receptors, therapeutic antibodies, cell growth, or stimulatory factors such as GCSF (Neupogen), epogen. In most preferred embodiments, agents that benefit from the methods of the invention are PEGylated forms or depot forms.

The present invention provides methods for administering antineoplastic agents. Such antineoplastic agents include a variety of agents including cytokines, angiogenesis inhibitors, classic anticancer agents and therapeutic antibodies. Cytokines immunomodulating agents and hormones that may be used in accordance with the invention include, but are not limited to interferons, interleukins (IL-1, -2, -4, -6, -8, -12) and cellular growth factors.

Angiogenesis inhibitors that can be used in the methods and compositions of the invention include but are not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); Fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-IC11; Neovastat; NM-3; Panzem; PI-88; Placental ribonuclease inhibitor; Plasminogen activator inhibitor; Platelet factor-4 (PF4); Prinomastat; Prolactin 16 kD fragment; Proliferin-related protein (PRP); PTK 787/ZK 222594; Retinoids; Solimastat; Squalamine; SS 3304; SU 5416; SU6668; SU 11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; ZD 6474; farnesyl transferase inhibitors (FTI); and bisphosphonates.

Other anti-cancer agents that can be used in accordance with the methods of invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukins (including recombinant interleukin 12, or rIL12, interferon alfa-2a; interferon alfa-2b; interferon alfa-n1; interferon alfa-n3; interferon beta-Ia; interferon gamma-Ib; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone BI; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovomm.

Other examples of antineoplastic agents that may be administered in accordance with the methods of the invention include therapeutic antibodies including but not limited to ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Smart M195 which is a humanized anti-CD33 IgG antibody (Protein Design Lab/Kanebo); LYMPHOCIDE™ which is a humanized anti-CD22 IgG antibody (Immunomedics); ICM3 is a humanized anti-ICAM3 antibody (ICOS Pharm); IDEC-114 is a primatied anti-CD80 antibody (IDEC Pharm/Mitsubishi); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

As used herein, and unless otherwise specified, the term “modulating circulation half life” means that increasing or decreasing the circulation half life of a therapeutic agent, which results in longer or shorter period duration of activity of that therapeutic agent, respectively. In this invention, circulation half life of a therapeutic agent can be modulated by varying the ratio between particulate and solution forms of the therapeutic agent to be delivered using methods of the invention in the mixture containing both forms. In principle, the higher the ratio between particulate and solution forms, the longer the circulation half life becomes. The desired circulation half life of a particular agent can be readily achieved by those of ordinary skill in the art using methods of the invention, as well as those well-known in the art. The circulation half life of a therapeutic agent can be determined using any methods known in the art, as well as those described herein.

5.1. Insulin Formulations

The invention encompasses methods of administering solution forms of insulin, particulate forms of insulin and mixture thereof, including fast-acting, intermediate-acting, and long-acting insulin formulations that may be obtained from any species or generated by any recombinant DNA technology known to one skilled in the art or any other method of creating new insulin analogs. Table 1 provides a non-limiting example of insulin formulations available and their mode of action, all of which are encompassed within the instant invention. The insulin formulations used in the methods and formulations of the invention may be a mixture of one or more insulin formulations.

The invention encompasses methods of administering solution forms of insulin (e.g., Humalog®) particulate forms of insulin (e.g., Humalog® Mix 50/50™, and mixtures thereof. The insulin formulations may be in different physical association states, including but not limited to monomeric, dimeric and hexameric states. The chemical state of insulin may be modified by standard recombinant DNA technology to produce insulin of different chemical formulas in different association states. Alternatively solution parameters, such as pH and Zn content, may be altered to result in formulations of insulin in different association states. Other chemical, biochemical or genetic modifications of insulin are also encompassed by the instant invention.

For therapeutic purposes doses and concentrations of insulin are expressed in units (U). One unit of insulin is equal to the amount required to reduce the concentration of blood glucose in a fasting rabbit to 45 mg/dL (2.5 mM). The current international standard is a mixture of bovine and porcine insulins and contains 24 U/mg. Homogenous preparations of insulin contain between 25 and 30 U/mg. Typically most commercial preparations of insulin are supplied in solution or suspension at a concentration of 100 U/mL (0.6 mM). The invention encompasses administering 1 to 50 U, preferably at least 10 U, most preferably 50 U of insulin to the intradermal space, preferably the papillary dermis. Using the methods of the invention lower doses of insulin are required to achieve a similar therapeutic efficacy as conventional methods of insulin therapy. The insulin formulations delivered in accordance with the methods of the invention are particularly effective in decreasing serum glucose levels and have improved therapeutic efficacy compared to the conventional methods for treating and/or preventing diabetes mellitus.

Formulations of insulin may be from different animal species including, limited but not to, swine, bovine, ovine, equine, etc. The chemical state of insulin may be modified by standard recombinant DNA technology to produce insulin of different chemical formulas in different association states. Alternatively solution parameters, such as pH and Zn content, may be altered to result in formulations of insulin in different association states. Formulations of insulins as commercially available are typically solutions of regular crystalline zinc insulin dissolved in a buffer at neutral pH. These preparations have rapid onset, e.g., 0.3-0.7 hours but a short duration of action, e.g., 5-8 hours. A non-limiting example of insulin formulations are Humulin R® (Lilly & Company) Novolin R®, Actrapid, Velosulin, Semilente. The kinetics of absorption of Semilente and regular insulin are similar, however Semilente has a longer duration of action, i.e., 12-16 hours. Over the past few years, there has been increased use of the very short acting insulin analogs, Lispro (Humalog®) and Aspart (NovoRapid®), which have even shorter times to onset and peak, but even shorter durations of action. Other preparations that are most frequently used are neutral protamine Hagedorn (NPH) insulin (isophane insulin suspension) and lente insulin (insulin zinc suspension). NPH insulin is a suspension of insulin in a complex with zinc and protamine in a phosphate buffer. Lente insulin is a mixture of crystallized and amorphous insulin in acetate buffer, which reduces the solubility of insulin. A non-limiting example of particulate or suspension insulin for formulations for use in the methods of the invention include NPH Iletin II, Lente Iletin II, Protaphane NPH, Lentard, Monotard, Mixtard, Humulin N, Novolin N, Novolin L, Humulin L, Humalog® Mix 50/50™, Humalog® NPL)

Administration of the very long acting insulins such as ultralente insulin (extended insulin zinc suspension) and protamine zinc insulin suspension and Glargine (Lantus®) are also encompassed by the invention. They have a very slow onset and a prolonged relatively “flat” peak of action. These insulins provide a low basal concentration of insulin through out the day. A non-limiting example of these formulations include ultralente Iletin I, PZI Iletin II.

TABLE 1
INSULIN FORMULATIONS
Properties of Insulin Preparations

| | | | ZINC | | | | |
|---|---|---|---|---|---|---|---|
| | | ADDED | CONTENT, | | ACTION, HOURS† | | |
| TYPE | APPEARANCE | PROTEIN | MG/100 U | BUFFER* | Onset | Peak | Duration |
| Rapid | | | | | | | |
| Lispro or | | Made by rDNA | | None | 0.1-0.5 | .75-1.5 | 4-6 |
| Aspart | | technology | | | | | |
| Regular | Clear | None | 0.01-0.04 | None or | 0.3-0.7 | 2-4 | 5.8 |
| (crystalline) | | | | phosphate | | | |
| Semilente | Cloudy | None | 0.2-0.25 | Acetate | 0.5-1.0 | 2-8 | 12-16 |
| Intermediate | | | | | | | |
| NPH (isophane) | Cloudy | Protamine | 0.016-0.04 | Phosphate | 1-2 | 6-12 | 18-24 |
| Lente | Cloudy | None | 0.2-0.25 | Acetate | 1-2 | 6-12 | 18-24 |
| Slow | | | | | | | |
| Ultralente | Cloudy | None | 0.2-0.25 | Acetate | 4-6 | 16-18 | 20-36 |
| Protamine zinc | Cloudy | Protamine | 0.2-0.25 | Phosphate | 4-6 | 14-20 | 24-36 |

| | | | | | |
|---|---|---|---|---|---|
| Glargine | Clear | Made by rDNA | 2-4 | 12 | 24 |
| | | technology | | | |

In some embodiments, the insulin formulations of the invention comprise a therapeutically effective amount of insulin and one or more other additives. Additives that may be used in the insulin formulations of the invention include for example, wetting agents, emulsifying agents, agents that change the quaternary structure of insulin or pH buffering agents. The insulin formulations of the invention may contain one or more other excipients such as saccharides and polyols. Additional examples of pharmaceutically acceptable carriers, diluents, and other excipients are provided in Remington's Pharmaceutical Sciences (Mack Pub. Co. N.J. current edition, all of which is incorporated herein by reference in its entirety.

The invention encompasses formulations in which insulin is in a particulate form, i.e., is not fully dissolved in solution. In some embodiments, at least 30%, at least 50%, at least 75% of the insulin is in particulate form. Although not intending to be bound by a particular mode of action, formulations of the invention in which insulin is in particulate form have at least one agent which facilitates the precipitation of insulin. Precipitating agents that may be employed in the formulations of the invention may be proteinacious, e.g., protamine, a cationic polymer, or non-proteinacious, e.g., zinc or other metals or polymers.

The form of insulin to be delivered or administered include solutions thereof in pharmaceutically acceptable diluents or solvents, emulsions, suspensions, gels, particulates such as micro- and nanoparticles either suspended or dispersed, as well as in-situ forming vehicles of the same. The insulin formulations of the invention may be in any form suitable for intradermal delivery. In one embodiment, the intradermal insulin formulation of the invention is in the form of a flowable, injectible medium, i.e., a low viscosity formulation that may be injected in a syringe or insulin pen. The flowable injectible medium may be a liquid. Alternatively the flowable injectible medium is a liquid in which particulate material is suspended, such that the medium retains its fluidity to be injectible and syringable, e.g., can be administered in a syringe. In a specific embodiment, the insulin formulation administered in accordance with the methods of the invention is Insulin Lispro (Eli Lilly & Company) at 100 U/mL. Preferably 1-50 U, most preferably 10 U, of Insulin Lispro are used in the methods of the invention. In another specific embodiment, the insulin formulation administered in accordance with the methods of the invention is 20 U 50% pre-mixed insulin Lispro (Humalog Mix 50/50™, containing 50% insulin lispro and 50% insulin lispro protamine suspension).

The intradermal insulin formulations of the present invention can be prepared as unit dosage forms. A unit dosage per vial may contain 0.1 to 0.5 mL of the formulation. In some embodiments, a unit dosage form of the intradermal formulations of the invention may contain 50 μL to 100 μL, 50 μL to 200 μL, or 50 μL to 500 μL of the formulation. If necessary, these preparations can be adjusted to a desired concentration by adding a sterile diluent to each vial. Insulin formulations administered in accordance with the methods of the invention are not administered in volumes whereby the intradermal space might become overloaded leading to partitioning to one or more other compartments, such as the SC compartment.

5.2. Administration of Insulin Formulation

In some embodiments, the present invention encompasses methods for intradermal delivery of insulin formulations described and exemplified herein to the intradermal compartment of a subject's skin, preferably by directly and selectively targeting the intradermal space, particularly the dermal vasculature, without entirely passing through it. Once the insulin formulation is prepared in accordance to the methods described supra, the formulation is typically transferred to an injection device for intradermal delivery, e.g., a syringe or insulin pen. The insulin may be in a commercial preparation, such as a vial or cartridge, specifically designed for intradermal injection. The insulin formulations of the invention are administered using any of the intradermal devices and methods known in the art or disclosed in WO 01/02178, published Jan. 10, 2002; and WO 02/02179, published Jan. 10, 2002.

The invention is based, in part, on the inventors' discovery that delivery of insulin formulations described and exemplified herein to the intradermal compartment, particularly the dermal vasculature provided for therapeutic and clinical efficacy for example for the treatment of diabetes. The insulin formulations of the invention have an improved absorption uptake within the intradermal space.

The actual method by which the intradermal administration of the insulin formulation is targeted to the intradermal space is not critical as long as it penetrates the skin of a subject to the desired targeted depth within the intradermal space without passing through it. In most cases, the device will penetrate the skin to a depth of about 0.5-2 mm. The invention encompasses conventional injection needles, catheters or microneedles of all known types, employed singularly or in multiple needle arrays. The dermal access means may comprise needle-less devices including ballistic injection devices. The terms “needle” and “needles” as used herein are intended to encompass all such needle-like structures with any bevel or even without a point. The term microneedles as used herein are intended to encompass structure 30 gauge and smaller, typically about 31-50 gauge when such structures are cylindrical in nature. Non-cylindrical structures encompass by the term microneedles would therefore be of comparable diameter and include pyramidal, rectangular, octagonal, wedged, and other geometrical shapes. They too may have any bevel, combination of bevels or may lack a point. The methods of the invention also include ballistic fluid injection devices, powder-jet delivery devices, piezoelectric, electromotive, electromagnetic assisted delivery devices, gas-assisted delivery devices, of which directly penetrate the skin to provide access for delivery or directly deliver substances to the targeted location within the dermal space.

Preferably however, the device has structural means for controlling skin penetration to the desired depth within the intradermal space. This is most typically accomplished by means of a widened area or hub associated with the shaft of the dermal-access means that may take the form of a backing structure or platform to which the needles are attached. The length of microneedles as dermal-access means are easily varied during the fabrication process and are routinely produced in less than 2 mm length. Microneedles are also a very sharp and of a very small gauge, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of substance delivered in a given period of time. The needle may eject its substance from the end, the side or both. Microneedles may be incorporated into a variety of devices such as holders and housings that may also serve to limit the depth of penetration. The dermal-access means of the invention may also incorporate reservoirs to contain the substance prior to delivery or pumps or other means for delivering the drug or other substance under pressure. Alternatively, the device housing the dermal-access means may be linked externally to such additional components.

The intradermal methods of administration comprise microneedle-based injection and infusion systems or any other means to accurately target the intradermal space. The intradermal methods of administration encompass not only microdevice-based injection means, but other delivery methods such as needle-less or needle-free ballistic injection of fluids or powders into the intradermal space, Mantoux-type intradermal injection, enhanced ionotophoresis through microdevices, and direct deposition of fluid, solids, or other dosing forms into the skin.

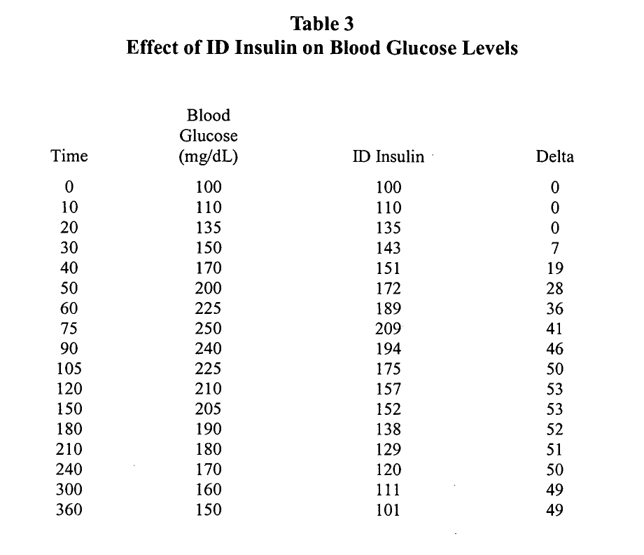

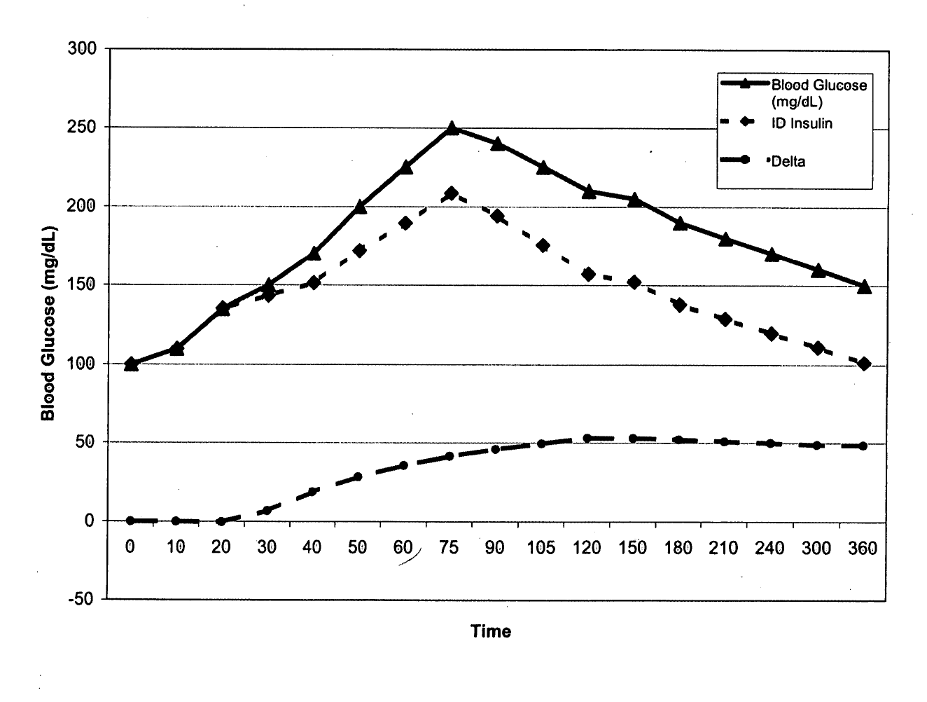

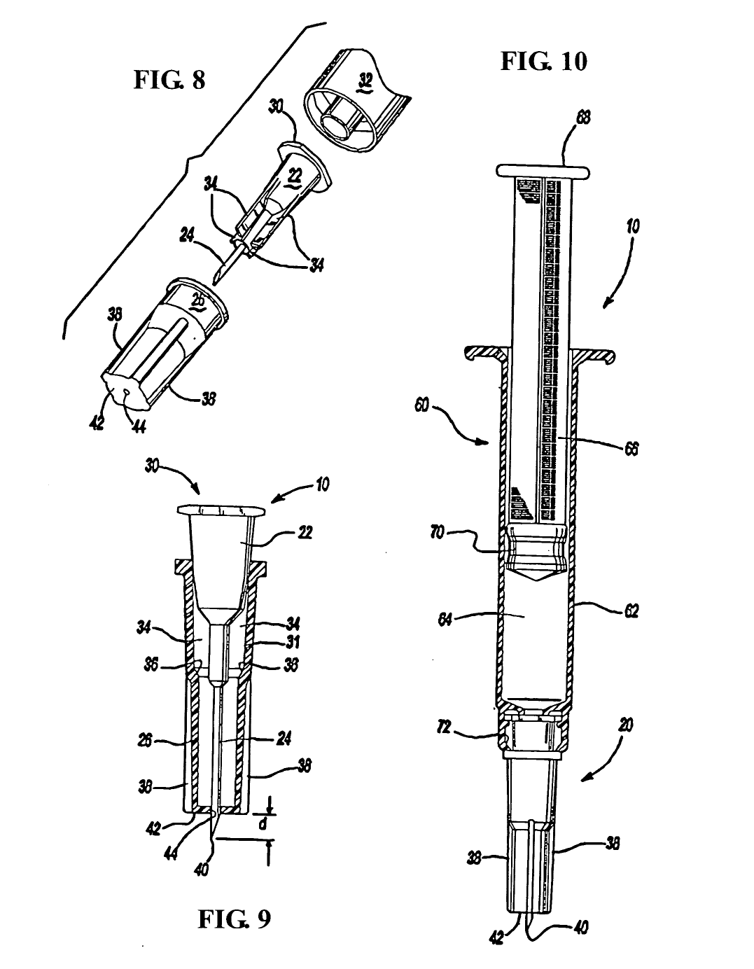

In particular embodiments, the formulations of the invention are administered using devices such as those exemplified in FIGS. 8-10, including a needle cannula having a forward needle tip and the needle cannula being in fluid communication with a substance contained in the drug delivery device and including a limiter portion surrounding the needle cannula and the limiter portion including a skin engaging surface, with the needle tip of the needle cannula extending from the limiter portion beyond the skin engaging surface a distance equal to approximately 0.5 mm to approximately 3.0 mm and the needle cannula having a fixed angle of orientation relative to a plane of the skin engaging surface of the limiter portion, inserting the needle tip into the skin of an animal and engaging the surface of the skin with the skin engaging surface of the limiter portion, such that the skin engaging surface of the limiter portion limits penetration of the needle cannula tip into the dermis layer of the skin of the animal, and expelling the substance from the drug delivery device through the needle cannula tip into the skin of the animal.

In a specific embodiment, the insulin formulations of the invention are administered to an intradermal compartment of a subject's skin, preferably the dermal vasculature using an intradermal Mantoux type injection, see, e.g., Flynn et al., 1994, Chest 106: 1463-5, which is incorporated herein by reference in its entirety. In a specific embodiment, the insulin formulation of the invention is delivered to the intradermal compartment of a subject's skin using the following exemplary method. The insulin formulation as prepared in accordance to methods disclosed in Section 5.1, is drawn up into a syringe, e.g., a 1 mL latex free syringe with a 20 gauge needle; after the syringe is loaded it is replaced with a 30 gauge needle for intradermal administration. The skin of the subject, e.g., mouse, is approached at the most shallow possible angle with the bevel of the needle pointing upwards, and the skin pulled tight. The injection volume is then pushed in slowly over 0.1-10 seconds forming the typical “bleb” and the needle is subsequently slowly removed. Preferably, only one injection site is used. In another specific embodiment, the insulin is stored in a cartridge and placed into a specific insulin pen. A micro-penneedle of 30-34 gauge is then placed into the septum of the cartridge and used in a method identical to the previous embodiment

By “improved pharmacokinetics” it is meant that an enhancement of pharmacokinetic profile is achieved as measured, for example, by standard pharmacokinetic parameters such as time to maximal plasma concentration (Tₘₐₓ), the magnitude of maximal plasma concentration (Cₘₐₓ) or the time to elicit a minimally detectable blood or plasma concentration (T_{lag}). By enhanced absorption profile, it is meant that absorption is improved or greater as measured by such pharmacokinetic parameters. The measurement of pharmacokinetic parameters and determination of minimally effective concentrations are routinely performed in the art. Values obtained are deemed to be enhanced by comparison with a standard route of administration such as, for example, subcutaneous administration or intramuscular administration. In such comparisons, it is preferable, although not necessarily essential, that administration into the intradermal layer and administration into the reference site such as subcutaneous administration involve the same dose levels, i.e., the same amount and concentration of drug as well as the same carrier vehicle and the same rate of administration in terms of amount and volume per unit time. Thus, for example, administration of a given pharmaceutical substance into the dermis at a concentration such as 100 μg/ml and rate of 100 μL per minute over a period of 5 minutes would, preferably, be compared to administration of the same pharmaceutical substance into the subcutaneous space at the same concentration of 100 μg/ml and rate of 100 μL per minute over a period of minutes.

The above-mentioned PK and PD benefits are best realized by accurate direct targeting of the dermal capillary beds. This is accomplished, for example, by using microneedle systems of less than about 250 micron outer diameter, and less than 2 mm exposed length. Such systems can be constructed using known methods of various materials including steel, silicon, ceramic, and other metals, plastic, polymers, sugars, biological and or biodegradable materials, and/or combinations thereof.

It has been found that certain features of the intradermal administration methods provide clinically useful PK/PD and dose accuracy. For example, it has been found that placement of the needle outlet within the skin significantly affects PK/PD parameters. The outlet of a conventional or standard gauge needle with a bevel has a relatively large exposed height (the vertical rise of the outlet). Although the needle tip may be placed at the desired depth within the intradermal space, the large exposed height of the needle outlet causes the delivered substance to be deposited at a much shallower depth nearer to the skin surface. As a result, the substance tends to effuse out of the skin due to backpressure exerted by the skin itself and to pressure built up from accumulating fluid from the injection or infusion and to leak into the lower pressure regions of the skin, such as the subcutaneous tissue. That is, at a greater depth a needle outlet with a greater exposed height will still seal efficiently where as an outlet with the same exposed height will not seal efficiently when placed in a shallower depth within the intradermal space. Typically, the exposed height of the needle outlet will be from 0 to about 1 mm. A needle outlet with an exposed height of 0 mm has no bevel and is at the tip of the needle. In this case, the depth of the outlet is the same as the depth of penetration of the needle. A needle outlet that is either formed by a bevel or by an opening through the side of the needle has a measurable exposed height. It is understood that a single needle may have more than one opening or outlets suitable for delivery of substances to the dermal space.

It has also been found that by controlling the pressure of injection or infusion the high backpressure exerted during ID administration can be overcome. By placing a constant pressure directly on the liquid interface a more constant delivery rate can be achieved, which may optimize absorption and obtain the improved pharmacokinetics. Delivery rate and volume can also be controlled to prevent the formation of wheals at the site of delivery and to prevent backpressure from pushing the dermal-access means out of the skin and/or into the subcutaneous region. The appropriate delivery rates and volumes to obtain these effects may be determined experimentally using only ordinary skill. Increased spacing between multiple needles allows broader fluid distribution and increased rates of delivery or larger fluid volumes. In addition, it has been found that ID infusion or injection often produces higher initial plasma levels of insulin than conventional SC administration. This may allow for smaller doses of insulin to be administered via the ID route.

The administration methods useful for carrying out the invention include both bolus and infusion delivery of insulin to humans or animals subjects. A bolus dose is a single dose delivered in a single volume unit over a relatively brief period of time, typically less than about 10 minutes. Infusion administration comprises administering a fluid at a selected rate that may be constant or variable, over a relatively more extended time period, typically greater than about 10 minutes. To deliver a substance the dermal-access means is placed adjacent to the skin of a subject providing directly targeted access within the intradermal space and the substance or substances are delivered or administered into the intradermal space where they can act locally or be absorbed by the bloodstream and be distributed systematically. The dermal-access means may be connected to a reservoir containing the substance or substances to be delivered.

Delivery from the reservoir into the intradermal space may occur either passively, without application of the external pressure or other driving means to the substance or substances to be delivered, and/or actively, with the application of pressure or other driving means. Examples of preferred pressure generating means include pumps, syringes, insulin pens, elastomer membranes, gas pressure, piezoelectric, electromotive, electromagnetic or osmotic pumping, or Belleville springs or washers or combinations thereof. If desired, the rate of delivery of the substance may be variably controlled by the pressure-generating means. As a result, the substance enters the intradermal space and is absorbed in an amount and at a rate sufficient to produce a clinically efficacious result.

As used herein, the term “clinically efficacious result” is meant a clinically useful biological response including both diagnostically and therapeutically useful responses, resulting from administration of a insulin. For example, diagnostic testing or prevention or treatment of a disease or condition is a clinically efficacious result. Such clinically efficacious results include diagnostic results such as the measurement of glomerular filtration pressure following injection of insulin,

5.3. Determination of Therapeutic Efficacy

The therapeutic efficacy of insulin formulations of the invention may be determined using any standard method known to one skilled in the art or described herein. The assay for determining the therapeutic efficacy of the insulin formulations of the invention may be in vivo or in vitro based assays, including animal based assays. Preferably, the therapeutic efficacy of the formulations of the invention is done in a clinical setting.

In some embodiments, the pharmacokinetics and pharmacodynamic parameters of insulin delivery is determined, preferably quantitatively using standard methods known to one skilled in the art. In preferred embodiments, the pharmacodynamic and pharmacokinetic properties of insulin delivery using the methods of the invention are compared to other conventional modes of insulin delivery, e.g., SC delivery, to establish the therapeutic efficacy of insulin administered in accordance with the methods of the invention. Pharmacokinetic parameters that may be measured in accordance with the methods of the invention include but are not limited to Tₘₐₓ, Cₘₐₓ, T_{lag}, AUC, etc. In specific embodiments, the pharmacokinetic parameters determined are maximal serum insulin Lispro concentrations (INSₘₐₓ), time to INSmax (TINSmax), Area under the glucose infusion rates in defined time-intervals (e.g., AUCIns 0-0.5 h, AUCIns 0-1 h, AUCIns 0-2 h, AUCIns 0-4 h, AUCIns 0-6 h), and C-peptide concentrations. Other pharmacokinetic parameters that may be measured in the methods of the invention include for example, half-life (t_{1/2}), elimination rate constant and partial AUC values.

Standard statistical tests which are known to one skilled in the art may be used for the statistical analysis of the pharmacokinetic and pharmacodynamic parameters obtained. The variables to be analyzed include for example pharmacodynamic measurements (based on the glucose infusion rates obtained), and serum C-peptide concentrations and pharmacokinetic measurements (based on the serum insulin Lispro concentrations).

The primary pharmacodynamic endpoint that may be measured under glucose clamp conditions is the area under the glucose infusion rates curve (AUC_{GIR}) in the two hours after insulin administration (AUC_{GIR} 0-2 h). Another pharmacodynamic endpoint that may be measured is the overall decrease in blood glucose over time may also be measured. For pharmacodynamic assessment the following parameters may be calculated: Maximal glucose infusion rate (GIRₘₐₓ), time to GIRₘₐₓ (TGIRₘₐₓ), Area under the glucose infusion rates in defined time-intervals (AUC_{GIR} 0-1 h, AUC_{GIR} 0-2 h, AUC_{GIR} 0-4 h, AUC_{GIR} 0-6 h), time to early and late half-maximal glucose infusion rate (early and late TGIR_{50%}).

Glucose infusion rates (GIR) registered after administration by two different routes, e.g., ID and SC, may be used to evaluate pharmacodynamic parameters. From these measurements, the area under the glucose infusion rate versus time curve from 0-6 hours (and other time intervals), the maximal glucose infusion rate, and time to the maximal glucose infusion rate may be determined. For the estimation of the pharmacodynamic summary measures fitting of a polynomial function to the GIR profile might be used. Other parameters, such as cumulative glucose infused over given intervals, may be determined.

An exemplary method for determining the pharmacokinetics and pharmacodynamic parameters of insulin delivery in accordance with the methods of the invention is the glucose clamp technique, see, e.g., DeFronzo et al., 1979, Am. J. Physiol. 237: 214-223; which is incorporated herein by reference in its entirety. Briefly the glucose clamp technique uses negative feedback from frequent blood glucose sample values to adjust a glucose infusion to maintain euglycemia. The glucose infusion rate therefore becomes a measure of the pharmacodynamic effect of any administered insulin.

In a specific embodiment, the invention encompasses determining the therapeutic efficacy of insulin Lispro administered in accordance with the methods of the invention by comparing the pharmacokinetic profile to that of SC delivery. An exemplary assay for determining the therapeutic efficacy of insulin Lispro may comprise the following: administering insulin Lispro (e.g., 10 U of 100 U/mL) with a 31G, 1.25 mm needle; or a 31G, 1.5 mm needle, with a 31G, 1.75 mm needle, or SC to humans. Preferably an 8 hour glucose clamp technique is used to maintain the euglycemic condition, wherein the wash out period between the clamps may be 3-20 days. Samples may be collected for determination of serum insulin Lispro concentrations and C-peptide levels and concentrations. Preferably sampling will occur from two hours before dosing and will continue for six hours after the dose is administered. Serum concentration of insulin Lispro and C-peptide may be determined using any method known to one skilled in the art, such as a radioimmunoassay. The blood samples are preferably centrifuged at 3000 rpm for a period of at least fifteen minutes at a temperature between 2 to 8° C., within one hour of sample collection. The serum from the collection tube is transferred for analysis of serum levels. Glucose infusion rates from the glucose clamp procedure may be monitored. The euglycemic clamp procedure should preferably last 6 hours for stabilization of blood glucose concentrations at the desired clamp level (e.g., at least 12 hours for testing long acting insulin).

Any injection site for intradermal administration may be used in the methods of the invention, including, but not limited to, the dermal region of thigh, abdomen, pectoral or chest deltoid, forearm and back of the forearm.

The invention encompasses any method known in the art for measuring fasting plasma glucose levels (FPGs) and non-fasting FPGs. FPGs are typically maintained within target levels as specified by guidelines provided by the American Diabetes Association (ADA) and the World Health Organization (WHO) (See, e.g., DCCT Res. Group, New England J. Med, 1993, 329: 977-86; and Kannel et al., 1979, Circulation, 59: 8-13 which are incorporated herein by reference in their entireties). FPGs and premeal glucose measurements are determined using standard methods known to one skilled in the art and are encompassed within the methods of the invention. In some embodiments, average glucose values over time are determined by measuring Hemoglobin A_{1c} levels (HbA_{1c}), which is a measure of the degree to which hemoglobin is glycosylated in erythrocytes and is expressed as a percentage of total hemoglobin concentration. HbA_{1c} levels reflect the exposure of erythrocytes to glucose in an irreversible and time and concentration dependent manner and provide an indication of the average blood glucose, concentration during the preceding 2-3 months, incorporating both pre and post prandial glycemia.

Any method known in the art for measuring PPG is encompassed within the methods of the invention. Such methods are known to one skilled in the art, see, e.g., Zimmerman, 2001, Am. J. Cardiol. 88 (Suppl): 32H-36H; American Diabetes Association, 2001, Diabetes Care, 24(4): 775-8; Verges et al., 2002, Diab. Nutr. Metab 15 (Suppl.): 28-32; all of which are incorporated herein by reference in their entireties). Preferably, PPG levels are determined within 1 hour after a meal, more preferably within 90 minutes, and most preferably within 2 hours.

The guidelines for target FPGs and PPGs are provided by ADA and WHO, and thus one skilled in the art practicing the methods of the invention would be able to determine the target desired levels in accordance with the methods of the invention. See, e.g., DCCT Res. Group, New England J. Med, 1993, 329: 977-86; and Kannel et al., 1979 Circulation, 59: 8-13. The ADA guidelines for example require the target FPG measurements to be <120 mg/dL (6.7 mmol/L) and HbA_{1c} levels <7%; 2 hr PPG levels <180 mg/dL (<10 mmol/L). Other guidelines from the EASD and AACE require the 2 hr PPG to be<140 mg/dL and the HbA_{1c} levels to be<6.5%.

5.4. Prophylactic and Therapeutic Uses

The invention provides methods of treatment and/or prevention which involve administering an insulin formulation to a subject, preferably a mammal, and most preferably a human for treating, managing or ameliorating symptoms associated with diabetes mellitus. The methods of the invention are useful for the treatment and/or prevention of diabetes or any related condition. The subject is preferably a mammal such as a non-primate, e.g., cow, pig, horse, cat, dog, rat, and a primate, e.g., a monkey such as a Cynomolgous monkey and a human. In a preferred embodiment, the subject is a human.

The diabetes and diabetes-related conditions which may be treated by the methods and formulations of the invention include, but are not limited to, diabetes characterized by the presence of elevated blood glucose levels, for example, hyperglycemic disorders such as diabetes mellitus, including both type 1, type 2 and gestational diabetes as well as other hyperglycemic related disorders such as obesity, increased cholesterol, kidney related disorders, cardiovascular disorders and the like. Other forms of diabetes mellitus that may be treated and/or prevented using the methods and formulations of the invention include for example, maturity onset diabetes of youth, insulinopathies, diabetes associated with other endocrine diseases (such as Cushing's syndrome, acromegaly, glucagonoma, primary aldosteronesim, insulin-resistant diabetes associated with acanthosis nigicans, lipoatrophic diabetes, diabetes induced by β-cell toxins, tropical diabetes, e.g., chronic pancreatitis associated with nutritional or toxic factors, diabetes secondary to pancreatic disease or surgery, diabetes associated with genetic syndrome, e.g., Prader-Willi Syndrome, diabetes secondary to endocrinopathies. Other diabetes-like conditions that may be treated using the methods of the invention include states of insulin resistance, with or without elevations in blood glucose, such as the metabolic syndrome that is associated with hypertension, lipid abnormalities and cardiovascular disease or polycystic ovarian syndrome.

The methods of the invention may be employed to, for example, lower glucose levels, improve glucose tolerance, increase hepatic glucose utilization, normalize blood glucose levels, stimulate hepatic fatty acid oxidation, reduce hepatic triglyceride accumulation, normalize glucose tolerance, treat or prevent insulin resistance. As used herein, “normalize” means to reduce the blood glucose level to an acceptable or average range for a healthy individual, which means within 10%, preferably 8%, more preferably 5% of the normal average blood glucose level for the subject.

The methods of the invention have an enhanced therapeutic efficacy in the treatment and management of one or more pathophysiological states associated with diabetes and related conditions. Pathophysiological conditions that may be improved using the methods and formulations of the invention include but are not limited to hyperglycemia, large vessel disease, microvascular disease, neuropathy, and ketoacidosis. Hyperglycemia as used herein carries its ordinary and customary meaning in the art and refers to abnormally high blood glucose levels usually associated with diabetes. Hyperglycemia can result from a reduction in the level of insulin secretion and/or the inability of insulin to convert glucose into energy with the resultant associated alterations in lipid metabolism. Large vessel disease as used herein carries its ordinary and customary meaning in the art and refers to an increased incidence, earlier onset and increased severity of atherosclerosis in the intima and calcification in the media of the arterial wall. Microvascular disease as used herein refers to an abnormality of the basement membrane of the capillaries characterized by added layers and consequent increased thickness of the lamina. Neuropathy as used herein refers to segmental injury to the nerves, associated with demyelination and Schwann cell degeneration which involves the sensory and motor neurons, nerve roots, the spinal cord, and the autonomous nervous system. Ketoacidosis as used herein refers to accumulation of ketones due to depressed levels of insulin.

The methods and formulations of the invention are therapeutically effective in reducing or eliminating one or more symptoms associated with diabetes mellitus or related condition. Symptoms that may be reduced or eliminated in accordance with the methods of the invention include but are not limited to symptomatic hyperglycemia, which may cause, blurred vision, fatigue, nausea, bacterial and fungal infections; nephropathy; sensory polyneuropathy, which causes sensory deficits, numbness, tingling, paresthesias in the extremities, etc.; foot ulcers and joint problems.

The invention encompasses intradermal delivery of formulations described herein in combination with one or more other therapies known in the art for the treatment and/or prevention of diabetes or a related disorder including but not limited to current and experimental therapies known to one skilled in the art. In some embodiments the formulations of the invention may be administered in combination with a therapeutically or prophylactically effective amount of one or more other therapeutic agents for the treatment or prevention of diabetes or a related disorder. Examples of therapeutic agents for treatment or prevention of diabetes or a related disorder include but are not limited to, agents that decrease FPG levels and agents that decrease PPG levels. Examples of agents that decrease FPG levels include but are not limited to sulfonylureas (e.g., Glipizide), metformin, alpha-glucosidase inhibitors (e.g., Acarbose, Miglitol), Thiasolidinediones. Examples of agents that decrease PPG levels include but are not limited to Repaglinide, Netiglinidem, Pioglitazone, and Rosiglitazone.

In certain embodiments, a formulation of the invention is administered to a mammal, preferably a human, concurrently with one or more other therapeutic agents useful for the treatment of diabetes. The term “concurrently” is not limited to the administration of prophylactic or therapeutic agents at exactly the same time, but rather it is meant that a formulation of the invention and the other agent are administered to a mammal in a sequence and within a time interval such that the formulation of the invention can act together with the other agent to provide an increased benefit than if they were administered otherwise. For example, each prophylactic or therapeutic agent may be administered at the same time or sequentially in any order at different points in time; however, if not administered at the same time, they should be administered sufficiently close in time so as to provide the desired therapeutic or prophylactic effect. Each therapeutic agent can be administered separately, in any appropriate form and by any suitable route. In various embodiments, the prophylactic or therapeutic agents are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In preferred embodiments, two or more components are administered within the same time period.

In other embodiments, the prophylactic or therapeutic formulations are administered at about 2 to 4 days apart, at about 4 to 6 days apart, at about 1 week part, at about 1 to 2 weeks apart, or more than 2 weeks apart. In preferred embodiments, the prophylactic or therapeutic agents are administered in a time frame where both agents are still active. One skilled in the art would be able to determine such a time frame by determining the half life of the administered agents.

In certain embodiments, the prophylactic or therapeutic formulations of the invention are cyclically administered to a subject. Cycling therapy involves the administration of a first agent for a period of time, followed by the administration of a second agent and/or third agent for a period of time and repeating this sequential administration. Cycling therapy can reduce the development of resistance to one or more of the therapies, avoid or reduce the side effects of one of the therapies, and/or improves the efficacy of the treatment.

In certain embodiments, prophylactic or therapeutic formulations are administered in a cycle of less than about 3 weeks, about once every two weeks, about once every 10 days or about once every week. One cycle can comprise the administration of a therapeutic or prophylactic agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

### 6. EXAMPLES

6.1. A Comparison of the Pharmacodynamic and Pharmacokinetic Properties of Insulin Lispro Intradermally Injected with the BD Microneedle-System vs. Subcutaneously Injected Insulin Lispro in an Open-Labeled, Randomized, Five-Way Crossover Study in Healthy Male Subjects

The primary objective of this study was to compare the pharmacokinetic and pharmacodynamic effects of 10 U insulin Lispro (100 U/mL from Eli Lilly and Company) delivered using BD microneedle injection system to that delivered subcutaneously. Secondary objectives of the study were to assess the optimal needle length for intradermal delivery of insulin Lispro reflected by the relative bioavailability following microneedle injection as compared to subcutaneous delivery. Furthermore, the study was designed to determine the intra-subject reproducibility of the delivery systems.

Study Design: Ten healthy male volunteers were used in a randomized study. Each subject (age between 18 and 45 years, BMI <27 kg/m²) was randomized to a treatment sequence consisting of five different treatments: (a) 10 Units of insulin Lispro (100 U/mL from Eli Lilly and Company) with the 31 Ga, 1.25 mm needle; (b) 10 Units of insulin Lispro (100 U/mL from Eli Lilly and Company) with the 31 Ga, 1.5 mm needle; (c) 10 Units of insulin Lispro (100 U/mL from Eli Lilly and Company) with the 31 Ga, 1.75 mm needle; (d) 10 Units of insulin Lispro (100 U/mL from Eli Lilly and Company) with the 31 Ga, 1.5 mm needle; (e); 10 Units of insulin Lispro (100 U/mL from Eli Lilly and Company) injected subcutaneously.

All treatments were studied with an 8 hours glucose clamp procedure as discussed below (Also see, DeFronzo et al., 1979, Am. J. Physiol. 237: 214-223). The wash-out period between the clamps was 3-20 days. Euglycemic conditions were maintained after drug administration using a glucose clamp procedure. Samples were collected for determination of serum insulin lispro and C-peptide concentrations, the glucose infusion rates from the glucose clamp procedure were documented. All treatments were identical in their sample collections and monitoring period for all visits. The euglycemic clamp procedure after study drug administration lasted 6 hours (+2 h baseline period for stabilization of blood glucose concentrations at the desired clamp level).

The overall study design is illustrated below.

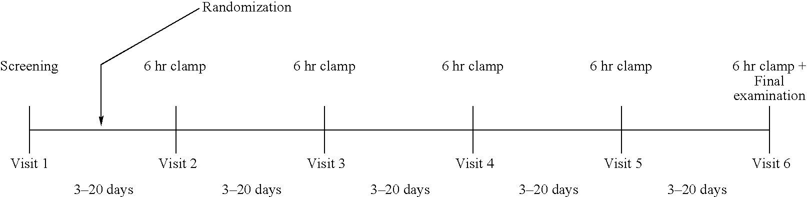

Materials and Supplies: The BD microneedle systems were manufactured under GMP compliance. The insulin used was available commercially as Insulin Lispro (100 U/mL from Eli Lilly and Company) in 3.0 ml cartridge and was purchased from a local pharmacy.

Dosage and Administration: Each subject received one of the possible ID treatments and the s.c. treatment at visit 2, 3, 4, 5 and visit 6 (as determined by the randomization sequence described above). The study drugs were given after an overnight fast of approximately 12 hours. The BD MicroneedleSystem administration was given in the morning following stabilization of the glucose clamp. The injection site was in the right upper quadrant of the right thigh. For BD Microneedle-System administration, the subject's thigh was cleaned with alcohol and allowed to dry. The microneedle was placed against the skin of the patient by an experienced health care professional and the 10 U of insulin Lispro injected intra-dermally. A successful injection will have no liquid visible above the skin and palpable fluid noted in the intra-dermal space. If there was significant fluid on the surface of the skin, the injection was considered unsuccessful and the session terminated for that day. The injection sites were blotted with a sponge which were weighed on a precision scale before and after this procedure. This was done to determine if there is any leakage from the site. Dosing was performed by an appropriately qualified member of the clinical unit designated by the investigator. If a subject was dropped from the study and replaced, then the new subject was assigned the same treatment sequence. The data from all subjects who complete at least one treatment were used in the analysis. After each dosing, safety, pharmacokinetic, and pharmacodynamic measures were evaluated. Due to the nature of the study this study was performed unblinded. For the duration of the study the chronic use of all agents which in the evaluation of the investigator would potentially interfere with the interpretation of trial results or known to cause clinically relevant interference with insulin action, glucose utilization or recovery from hypoglycemia was prohibited.

Pharmacodynamic Measurements: The subjects underwent five euglycemic clamp procedures on five separate days. The duration of each study period was approximately 9 hours. All clamp studies were performed after an overnight (approximately 12 hours) fast.

The Glucose Clamp Procedure: Subjects fasted (except for water) for approximately 12 hours prior to each treatment and until completion of the treatment period. Strenuous physical activity, smoking, and alcohol intake were not permitted for the 24 hours prior to each admission to the clinical research unit. On the morning of the treatment, subjects were not allowed to drink coffee, tea, or caffeine-containing beverages. The study started in the morning. A 17-gauge PTFE catheter was inserted into an antecubital vein for blood sampling for measurement of blood glucose, C-peptide and serum insulin lispro concentrations. The line was kept patent with 0.15-mmol/L (0.9%) sterile saline. A dorsal hand or a wrist vein of the same arm was cannulated in retrograde fashion for insertion of an 18-gauge PTFE double-lumen catheter, which was connected to the glucose sensor of a Biostator. The catheterized hand was warmed to an air temperature of approximately 55° C. On the contralateral arm, a third vein was cannulated with an 18-gauge PTFE catheter to infuse glucose (20% in water). In the same cannula insulin Huminsulin Normal (Regular Human Insulin), 100 U/mL from Eli Lilly and Company) was infused intravenously throughout the study with an infusion rate of 0.15 mU/kg/min to eliminate endogenous insulin secretion. This insulin does not interfere in the specific Lispro insulin assay. The target level for both glucose clamp experiments were 5 mmol/L. The clamp level was kept constant by a variable-rate intravenous infusion of 20% glucose. After insertion of the necessary venous lines the clamp level was kept constant automatically by the Biostator at the target value by varying the infusion rate of an intravenous glucose infusion. After a two-hour baseline period, at time-point 0, insulin Lispro was administered by the BD Microneedle-System or by subcutaneous injection. The pharmacodynamic response elicited by the study medication was studied (and documented) for another 6 hours. No food intake was allowed during this period but water could be consumed as desired.

Sample Size and Data Analysis Methods: A total of 10 subjects completed all 5 treatment days. Any subject who did not complete the five test visits was replaced. The sample size for this explorative study was selected to provide descriptive data. It is not the main aim of this study to find statistically significant differences between the forms of administrations. All comparisons were performed using Fisher exact test, (two-tailed) with a nominal significance level of 0.05; however, comparisons resulting in a p-value of less than 0.10 were also discussed as an indication of a difference. All confidence intervals were computed as two-sided, 95% confidence intervals.

Pharmacokinetic Analyses: For pharmacokinetic assessment the following parameters were calculated: Maximal serum insulin lispro concentrations (INSₘₐₓ), time to INSₘₐₓ(TINS), area under the insulin concentration versus time curve in defined time-intervals (AUC_{Ins 0-1 h}, AUC_{Ins 0-2 h}, AUC_{Ins 0-4 h}, AUC_{Ins 0-6 h}), and C-peptide concentrations. Parameters determined included also other pharmacokinetic parameters, such as half-life (t_{1/2}), elimination rate constant (λz) and other partial AUC values, may be calculated if considered appropriate. Parameters were calculated for each individual subject enrolled within the study. The primary analysis of this endpoint was to compare the intra subject variation of the two microneedle treatments. Comparison of the inter subject variation were a secondary analysis.

Pharmacodynamic Analyses: The primary pharmacodynamic endpoint was the area under the glucose infusion rates curve (AUC_{GIR}) in the two hours after drug administration (AUC_{GIR}^{_{0-2 h}}). For pharmacodynamic assessment the following parameters were calculated: Maximal glucose infusion rate (GIRₘₐₓ), time to GIRₘₐₓ (TGIRₘₐₓ), area under the glucose infusion rates in defined time-intervals (AUCGIR_{0-1 h}, AUCGIR_{0-2 h}, AUCGIR_{0.4 h}, AUCGIR_{0-6 h}) time to early and late half-maximal glucose infusion rate (early and late TGIR50%). Glucose infusion rates (GIR) registered after application by the two different routes were used to evaluate pharmacodynamic parameters. From these measurements, the area under the glucose infusion rate versus time curve from 0-6 hours (and other time intervals), the maximal glucose infusion rate, and time to the maximal glucose infusion rate were used. For the estimation of the pharmacodynamic summary measures fitting of a polynomial function to the GIR profile could be used. Standard statistical tests were used for the statistical analysis of the pharmacokinetic parameters obtained. If appropriate, a natural logarithmic transformation of the data was performed to ensure that the data are approximately normally distributed. Additional glucose measurements were analyzed as deemed appropriate, such as partial AUC values.

Results

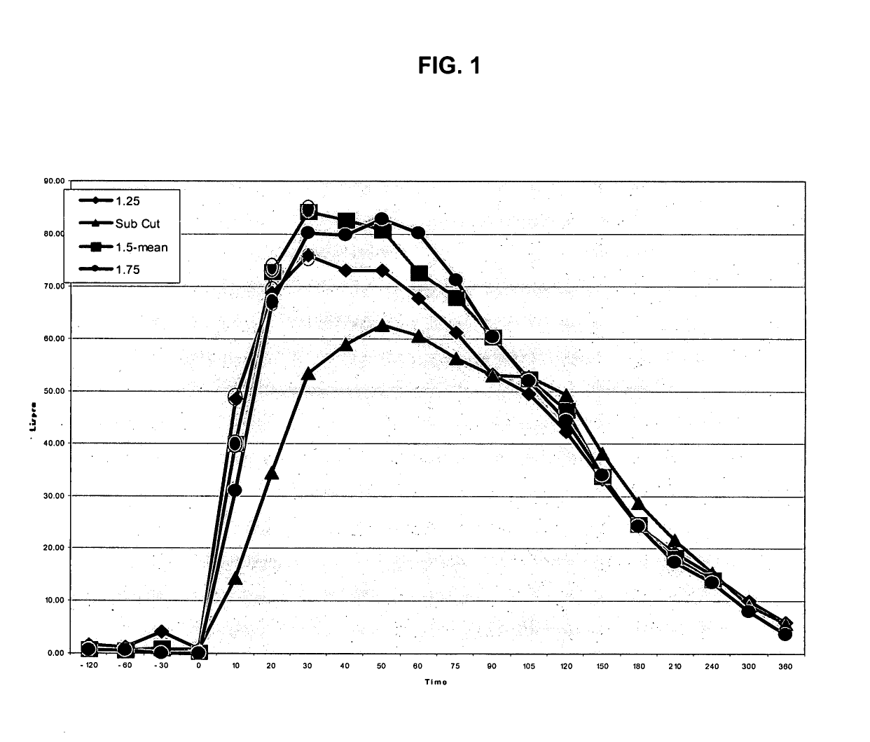

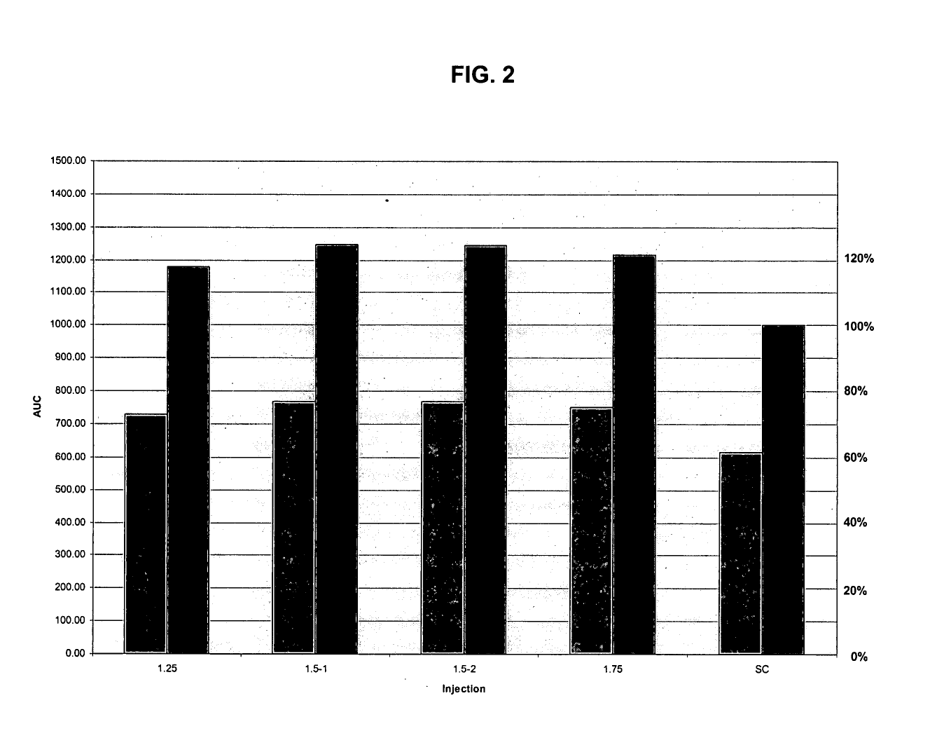

Insulin Lispro was injected intradermally with the BD Microneedle-System at varying depths, specifically at depth of 1.25 mm, 1.5 mm, and 1.75 mm. The pharmacokinetic and pharmacodynamic parameters of the insulin delivered ID were compared to delivery of insulin subcutaneously. The onset of systemically available insulin delivered ID is more rapid at all three depths as compared to SC (FIG. 1). The time to reach maximum concentration is shorter (Tₘₐₓ) and the maximum concentration obtained is higher for ID vs. SC. When the depth of injection is 1.75 mm or 1.5 mm, the highest Cₘₐₓ is obtained. Furthermore, there is a higher bioavailability of insulin upon ID delivery compared to SC delivery (FIGS. 1 and 2).

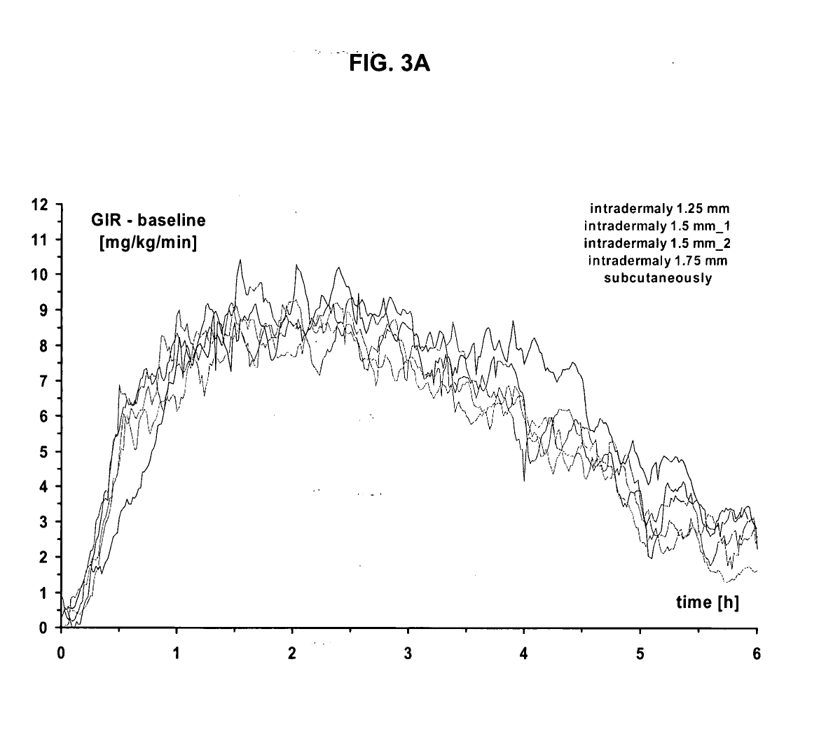

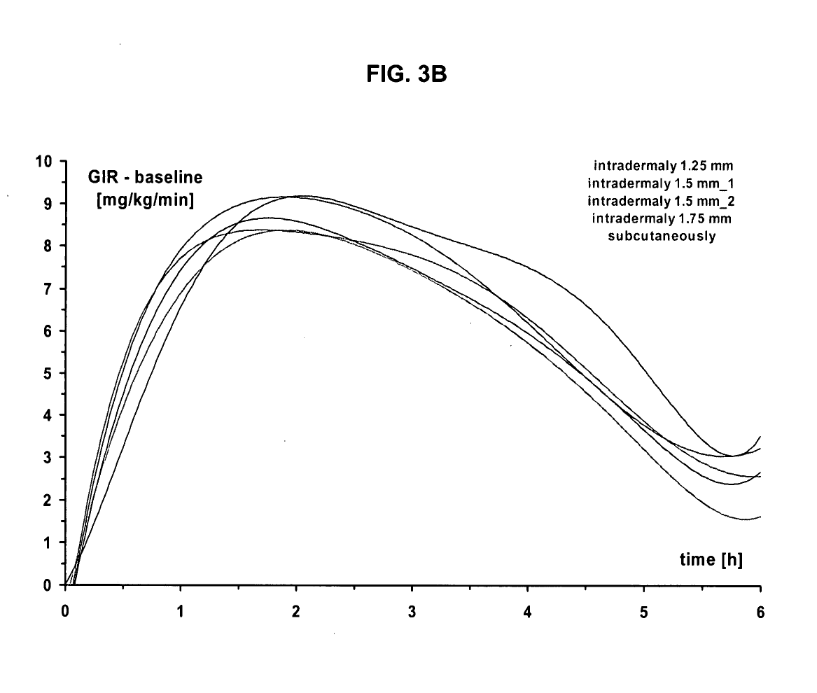

FIGS. 3A and B show the pharmacodynamic biological response to the administered insulin as measured by an increase in glucose infusion rate to compensate for the decrease in blood glucose due to the presence of insulin. ID delivery at all depths shows a faster and greater change in the blood glucose levels as measured by glucose infusion rate. Although the maximum glucose response levels, measured as the glucose infusion rate, were similar between ID and SC delivery.

6.2. A Comparison of the Pharmacodynamic and Pharmacokinetic Properties of a 50% Pre-Mixed Insulin Lispro (lispro 50% and Lispro-Protamine 50%) Intradermally Injected with the BD Microneedle-System vs. Subcutaneously Injected 50% Pre-Mixed Insulin Lispro in an Open-Labeled, Randomized, Three-Way Crossover Study in Healthy Male Subjects

The primary objective of this study was to compare the pharmacokinetic and pharmacodynamic effect of 20 U 50% pre-mixed insulin lispro (Humalog® Mix 50/50™, containing 50% insulin lispro and 50% insulin lispro protamine suspension in 100 U/mL (from Eli Lilly and Company) applied with a 1.5 mm BD Microneedle-Systems with that of 20 U 50% pre-mixed insulin Lispro applied subcutaneously.

Study Design: 10 healthy, male subjects were used in a randomized study. Each subject was randomized to a treatment sequence consisting of three different treatments: (a) 20 Units of 50% pre-mixed insulin lispro (Humalog® Mix 50/50™, containing 50% insulin lispro and 50% insulin lispro protamine suspension in 100 U/mL from Eli Lilly and Company) with the 31 Ga, 1.5 mm needle; (b) 20 Units of 50% pre-mixed insulin Lispro (Humalog® Mix50™, containing 50% insulin lispro and 50% insulin lispro protamine suspension in 100 U/mL from Eli Lilly and Company) injected subcutaneously; (c) 20 Units of 50% pre-mixed insulin lispro (Humalog® Mix 50/50™, containing 50% insulin lispro and 50% insulin lispro protamine suspension in 100 U/mL from Eli Lilly and Company) with the 31 Ga, 1.5 mm needle

All treatments were studied with a 12 hour glucose clamp procedure as described above. The wash-out period between the clamps was 3-20 days. Euglycemic conditions were maintained after drug administration using a glucose clamp procedure. Samples were collected for determination of serum insulin lispro and C-peptide concentrations, the glucose infusion rates from the glucose clamp procedure were documented. All treatments were identical in their sample collections and monitoring period for all visits. The euglycemic clamp procedure after study drug administration lasted 12 hours (+2 h baseline period for stabilization of blood glucose concentrations at the desired clamp level).

The overall study design is illustrated below.

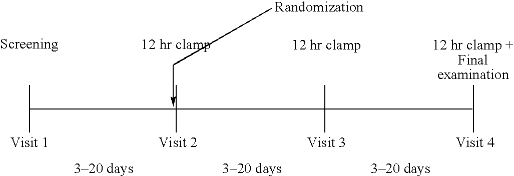

Administration and Sampling: Each subject received 3 injections in the thigh in a randomized fashion two injections were from a 1.5 mm, 31 Ga ID syringe in a bolus fashion (10-20 sec administration duration) and a control SC administration from a standard insulin syringe (30 G, 8 mm). The duplicate ID injection was designed to test intrasubject variability. Blood insulin and C-peptide levels were monitored for 12 hours post-administration, and quantified by standard clinical assay procedures. Blood glucose was maintained constant by IV glucose infusion during the 12 hours post insulin administration using a euglycemic glucose clamp. Increased glucose infusion rate (GIR) to maintain euglycemia due to insulin metabolic activity was recorded as the primary marker for pharmacodynamic effect. All other methods, including sampling, data analysis were done as described in the Example above.

Results:

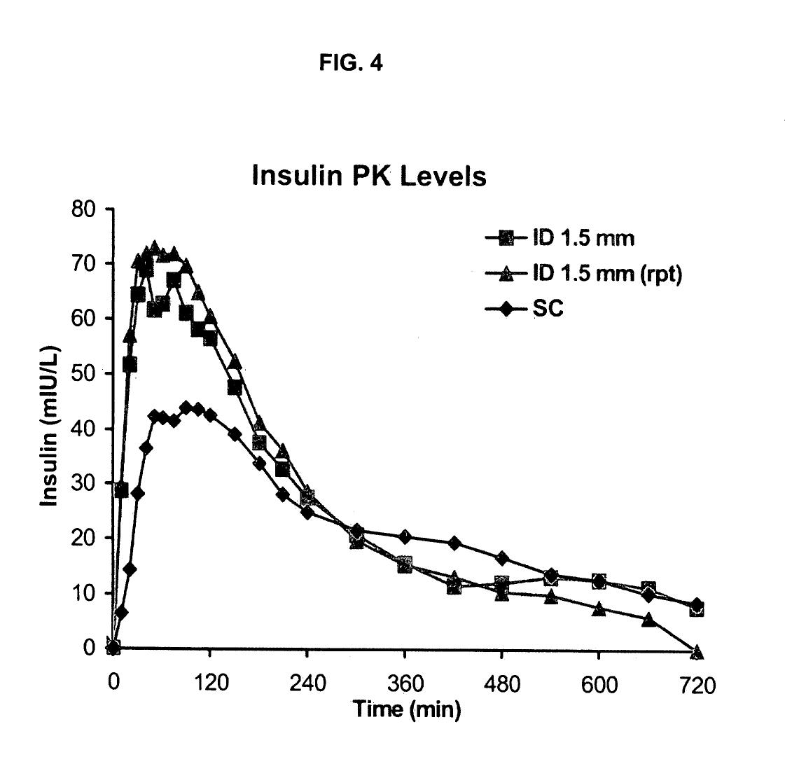

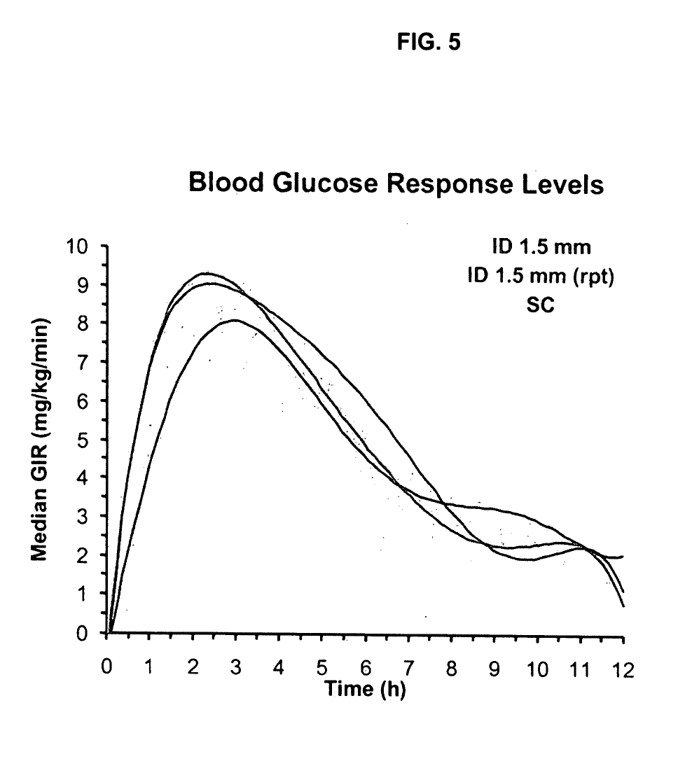

Graphs of mean plasma insulin levels and median GIR rates are shown in FIGS. 4 and 5. This study represents the pharmacokinetic (PK) and/or pharmacodynamic (PD) of particulates administered via ID administration. ID administration of Lispro mix exhibits similar effects to Lispro solution (as shown in Example 6.1), i.e., faster onset (shorter Tₘₐₓ), higher AUC (bioavailability), higher Cₘₐₓ. These results were unexpected because although the ID uptake mechanism seems to function for most solutions it was unclear whether it would do so with particulates. In spite of the rapid uptake, ID delivery still exhibits an extended duration of action out to 12 h. It is unclear if the extended duration activity is due to a localized dermal or other tissue depot or the slow dissolution of the insulin precipitate after uptake and systemic distribution. ID delivery does show a reduced PD effect at later time points (>8 h) indicating a reduction in late phase insulin activity vs SC delivery. This may have potential benefit for therapy by reducing the incidence of early morning hypoglycemia often encountered in diabetics on split mix therapy.

6.3. Effect of Intradermal Insulin Delivery on Post-Prandial Glucose

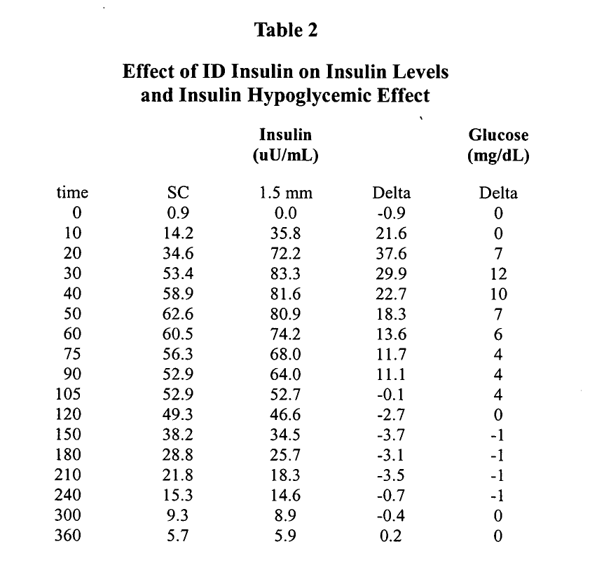

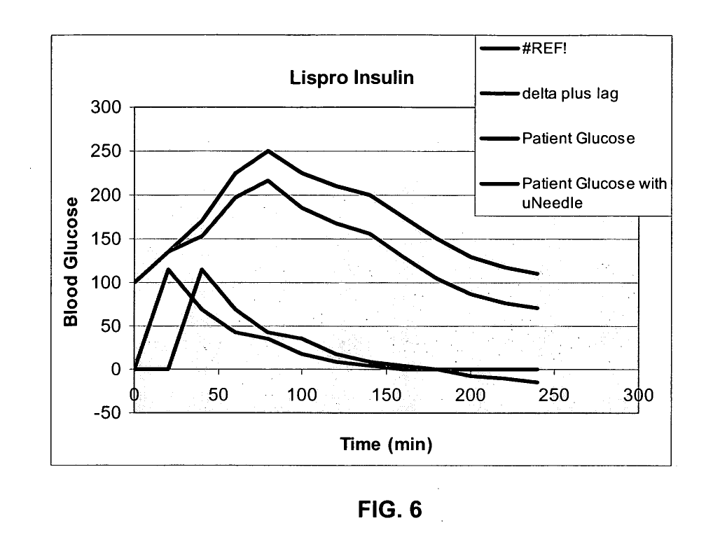

The primary objective of this analysis was to evaluate the effect of intradermal insulin delivery on post-prandial glucose levels. The analysis focused on the effect of intradermal delivery of 10 U insulin Lispro (100 U/mL from Eli Lilly and Company) delivered using BD microneedle injection system (at a depth of 1.5 mm) and compared to Insulin Lispro delivered subcutaneously. The data from Example 6.1 above was used to determine delta insulin which is the difference in the AUC of the insulin levels of the subjects who received Insulin via ID delivery with 1.5 mm microneedle and subcutaneous injection for the period indicated (e.g., 0-10 min “10”, 11-20 min “20”, etc.). To determine the effect of the delta insulin on the blood glucose in a patient using a microneedle, ISF or insulin sensitivity factor was determined. ISF was determined in insulin units (not AUC) and for Insulin Lispro, this is typically determined by the “rule of 1500”, i.e., dividing 1500 by the total daily insulin. For a typical patient with type 1 diabetes, the total daily insulin is about 60 U, so the ISF is 25 mg/dL/Unit insulin. From the data from Example 6.1, 10 Units of insulin produced an AUC of 780, i.e., 78 AUC units are equivalent to 1 insulin Unit. Thus, the ISF determined in AUC units was 0.33 mg/dL/AUC unit (see, the last column of Table 2). The ISF values were used to determine the amount of extra glucose lowering expected from the extra insulin. A 25 minute delay in action of insulin was utilized. FIG. 6 shows the insulin levels for the subcutaneous injection, the intradermal injection and the difference between the 2 modes of delivery.

Table 3 shows the effect of the additional insulin on the expected insulin levels in a patient with type 1 diabetes. The subcutaneous insulin column is the data that is often seen in patients with diabetes. After eating, glucose rises rapidly, peaks at 60-90 minutes, then as insulin acts, falls over the next few hours. The column labeled ID insulin takes account of the additional and earlier insulin action (last column of table 2) to predict the glucose lowering effect of the additional insulin. The effect on a glucose value measured at 2 hours would be about 60 mg/dL. The effect is plotted in FIG. 7.

As shown in FIGS. 6 and 7 (and the accompanied Tables 2 and 3), intradermal insulin delivery results in a 60% higher biopotency relative to subcutaneous insulin delivery within the first hour of delivery. Within the first hour, insulin is absorbed rapidly and constitutes 25% of the total insulin. Intradermal insulin delivery is thus effective in controlling PPG levels.

While the biopotency of insulin delivered ID as compared to SC delivery was measured over a six hour time period, the most dramatic increase of biopotency was observed within the first hour following ID administration (See FIG. 7). This dramatic increase of insulin biopotency when administered intradermally results in significant reductions of post-prandial glucose levels and tighter glycemic controls. Thus, intradermal delivery of insulin results in significant therapeutic advantages when compared to conventional routes of administration, e.g., subcutaneous delivery.

Accordingly, while the foregoing description and drawings represent embodiments of the present invention, it will be understood that various additions, modifications, and substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims, and not limited to the foregoing description.

1. A method for administration of an insulin formulation to a human subject, comprising delivering the insulin formulation into an intradermal compartment of the human subject's skin, so that the insulin formulation is deposited at a depth of 1.25 mm.

2. A method for administration of an insulin formulation to a human subject, comprising delivering the insulin formulation into an intradermal compartment of the human subject's skin, so that the insulin formulation is deposited at a depth of 1.5 mm.

3. A method for administration of an insulin formulation to a human subject, comprising delivering the insulin formulation into an intradermal compartment of the human subject's skin, so that the insulin formulation is deposited at a depth of 1.75 mm.

4. The method of any of claims 1-3 wherein the insulin formulation is in solution form.

5. The method of claim 4, wherein the insulin formulation is Humalog®.

6. The method of claim 4, wherein the insulin formulation is in particulate form.

7. The method of claim 6, wherein the insulin formulation is Humalog® Mix 50/50™.

8. The method of any of claims 1-3, wherein the onset of systemically available insulin delivered is more rapid compared to subcutaneous delivery.

9. The method of any of claims 1-3, wherein the method results in a faster and greater change in the blood glucose levels compared to subcutaneous delivery.

10. A method for administration of an insulin formulation to a human subject, comprising delivering the insulin formulation into an intradermal compartment of the human subject's skin, wherein the insulin formulation comprises a mixture of solution and particulate forms and wherein the particulate form is from about 1% to about 99% of the total formulation, so that the insulin formulation is deposited at a depth of 1.25 mm.

11. A method for administration of an insulin formulation to a human subject, comprising delivering the insulin formulation into an intradermal compartment of the human subject's skin, wherein the insulin formulation comprises a mixture of solution and particulate forms and wherein the particulate form is from about 1% to about 99% of the total formulation, so that the insulin formulation is deposited at a depth of 1.5 mm.

12. A method for administration of an insulin formulation to a human subject, comprising delivering the insulin formulation into an intradermal compartment of the human subject's skin, wherein the insulin formulation comprises a mixture of solution and particulate forms and wherein the particulate form is from about 1% to about 99% of the total formulation, so that the insulin formulation is deposited at a depth of 1.75 mm.

13. A method for administration of an insulin formulation in particulate form to a human subject, comprising delivering the insulin formulation into an intradermal compartment of the human subject's skin, so that the insulin formulation is deposited at a depth of 1.25 mm.

14. A method for administration of a insulin formulation in particulate form to a human subject, comprising delivering the insulin formulation into an intradermal compartment of the human subject's skin, so that the insulin formulation is deposited at a depth of 1.5 mm.

15. A method for administration of a insulin formulation in particulate form to a human subject, comprising delivering the insulin formulation into an intradermal compartment of the human subject's skin, so that the insulin formulation is deposited at a depth of 1.75 mm.

16. The method of any of claims 13-15, wherein the administered insulin has a lower Tₘₐₓ, a higher Cₘₐₓ, and a higher bioavailability, compared to subcutaneous delivery.

17. The method of any of claims 1-3, wherein the biopotency of insulin is increased by 60% compared to subcutaneous delivery.

18. The method of any of claims 1-3, wherein the insulin delivered results in reduction of post-prandial glucose levels by at least 20 mg/dL.

19. The method of any of claims 1-3, wherein the insulin delivered results in reduction of post-prandial glucose levels by at least 30 mg/dL.

20. The method of any of claims 1-3, wherein the insulin delivered results in reduction of post-prandial glucose levels by at least 45 mg/dL.

18. A method of eliciting a prolonged circulation of insulin in a human subject, comprising delivering into an intradermal compartment of the human subject's skin an insulin formulation which comprises both particulate and solution forms of insulin.

19. The method of claim 18, wherein the onset of systemically available insulin delivered is more rapid compared to subcutaneous delivery.

20. A method of modulating circulation half life of insulin in a human subject, comprising administering into an intradermal compartment of the human subject's skin a composition comprising both particulate and solution forms of insulin, wherein the ratio between the particulate and solution forms of the therapeutic agent is varied.

21. A method of modulating circulation half life of a therapeutic agent in a human subject, comprising administering into an intradermal compartment of the human subject's skin a composition comprising both particulate and solution forms of the therapeutic agent, wherein the ratio between the particulate and solution forms of the therapeutic agent is varied.

22. The method of claim 20 or 21, wherein the onset of systemically available therapeutic agent delivered is more rapid compared to subcutaneous delivery.

23. The method of claim 21, wherein the therapeutic agent is a protein.
